SEQUENCE LISTING (14 sequences)

SEQ ID NO: 1 - protein, 412 aa, Ricinus communis
FRQITKNQKKKVRKKTMALKLNPFLSQTQKLPSFALPPMASTRSPKFYMASTLKSGSKEVENLKKPFMPPREVHVQVTHSMPPQKIEIFKSLDNWAEENILVHLKPVEKCWQPQDFLPDPASDGFDEQVRELRERAKEIPDDYFVVLVGDMITEEALPTYQTMLNTLDGVRDETGASPTSWAIWTRAWTAEENRHGDLLNKYLYLSGRVDMRQIEKTIQYLIGSGMDPRTENSPYLGFIYTSFQERATFISHGNTARQAKEHGDIKLAQICGTIAADEKRHETAYTKIVEKLFEIDPDGTVLAFADMMRKKISMPAHLMYDGRDDNLFDHFSAVAQRLGVYTAKDYADILEFLVGRWKVDKLTGLSAEGQKAQDYVCRLPPRIRRLEERAQGRAKEAPTMPFSWIFDRQVKL

SEQ ID NO: 2 - protein, 363 aa, Ricinus communis
ASTLKSGSKEVENLKKPFMPPREVHVQVTHSMPPQKIEIFKSLDNWAEENILVHLKPVEKCWQPQDFLPDPASDGFDEQVRELRERAKEIPDDYFVVLVGDMITEEALPTYQTMLNTLDGVRDETGASPTSWAIWTRAWTAEENRHGDLLNKYLYLSGRVDMRQIEKTIQYLIGSGMDPRTENSPYLGFIYTSFQERATFISHGNTARQAKEHGDIKLAQICGTIAADEKRHETAYTKIVEKLFEIDPDGTVLAFADMMRKKISMPAHLMYDGRDDNLFDHFSAVAQRLGVYTAKDYADILEFLVGRWKVDKLTGLSAEGQKAQDYVCRLPPRIRRLEERAQGRAKEAPTMPFSWIFDRQVKL

SEQ ID NO: 3 - protein, 363 aa, Ricinus communis
ASTLKSGSKEVENLKKPFMPPREVHVQVTHSMPPQKIEIFKSLDNWAEENILVHLKPVEKCWQPQDFLPDPASDGFDEQVRELRERAKEIPDDYFVVLVGDMITEEALPTYQTMLNRLDGVRDETGASPTSWAIWTRAWTAEENRHGDLLNKYLYLSGRVDMRQIEKTIQYLIGSGMDPRTENSPYLGFIYTSFQERATFISHGNTARQAKEHGDIKLAQICGTIAADEKRHETAYTKIVEKLFEIDPDGTVLAFADMMRKKISMPAHLMYDGRDDNLFDHFSAVAQRLGVYTAKDYADILEFLVGRWKVDKLTGLSAEGQKAQDYVCRLPPRIRRLEERAQGRAKEAPTMPFSWIFDRQVKL

SEQ ID NO: 4 - protein, 363 aa, Ricinus communis
ASTLKSGSKEVENLKKPFMPPREVHVQVTHSMPPQKIEIFKSLDNWAEENILVHLKPVEKCWQPQDFLPDPASDGFDEQVRELRERAKEIPDDYFVVLVGDMITEEALPTYQTMLNTLDGVRDETGASPTSWAIWTRAWTAEENRHGDLLNKYLYLSGRVDMRQIEKTIQYLIGSGMDPRTENSPYLLFIYTSFQERATFISHGNTARQAKEHGDIKLAQICGTIAADEKRHETAYTKIVEKLFEIDPDGTVLAFADMMRKKISMPAHLMYDGRDDNLFDHFSAVAQRLGVYTAKDYADILEFLVGRWKVDKLTGLSAEGQKAQDYVCRLPPRIRRLEERAQGRAKEAPTMPFSWIFDRQVKL

SEQ ID NO: 5 - protein, 363 aa, Ricinus communis
ASTLKSGSKEVENLKKPFMPPREVHVQVTHSMPPQKIEIFKSLDNWAEENILVHLKPVEKCWQPQDFLPDPASDGFDEQVRELRERAKEIPDDYFVVLVGDMITEEALPTYQTMLNTLDGVRDETGASPTSWAIWTRAWTAEENRHGDLLNKYLYLSGRVDMRQIEKTIQYLIGSGMDPRTENSPYLGFIYTSFQERATFISHGNTARQAKEHGDIKLAQICGTIAADEKRHETAYTKIVEKLFEIDPDGTVLAFADMMRKKISMPAHLMYDGRDDNLFKHFSAVAQRLGVYTAKDYADILEFLVGRWKVDKLTGLSAEGQKAQDYVCRLPPRIRRLEERAQGRAKEAPTMPFSWIFDRQVKL

SEQ ID NO: 6 - protein, 363 aa, Ricinus communis
ASTLKSGSKEVENLKKPFMPPREVHVQVTHSMPPQKIEIFKSLDNWAEENILVHLKPVEKCWQPQDFLPDPASDGFDEQVRELRERAKEIPDDYFVVLVGDMITEEALPTYQTMLNRLDGVRDETGASPTSWAIWTRAWTAEENRHGDLLNKYLYLSGRVDMRQIEKTIQYLIGSGMDPRTENSPYLGFIYTSFQERATFISHGNTARQAKEHGDIKLAQICGTIAADEKRHETAYTKIVEKLFEIDPDGTVLAFADMMRKKISMPAHLMYDGRDDNLFKHFSAVAQRLGVYTAKDYADILEFLVGRWKVDKLTGLSAEGQKAQDYVCRLPPRIRRLEERAQGRAKEAPTMPFSWIFDRQVKL

SEQ ID NO: 7 - protein, 396 aa, Ricinus communis
MALKLNPFLSQTQKLPSFALPPMASTRSPKFYMASTLKSGSKEVENLKKPFMPPREVHVQVTHSMPPQKIEIFKSLDNWAEENILVHLKPVEKCWQPQDFLPDPASDGFDEQVRELRERAKEIPDDYFVVLVGDMITEEALPTYQTMLNTLDGVRDETGASPTSWAIWTRAWTAEENRHGDLLNKYLYLSGRVDMRQIEKTIQYLIGSGMDPRTENSPYLGFIYTSFQERATFISHGNTARQAKEHGDIKLAQICGTIAADEKRHETAYTKIVEKLFEIDPDGTVLAFADMMRKKISMPAHLMYDGRDDNLFDHFSAVAQRLGVYTAKDYADILEFLVGRWKVDKLTGLSAEGQKAQDYVCRLPPRIRRLEERAQGRAKEAPTMPFSWIFDRQVKL

SEQ ID NO: 8 - protein, 396 aa, Hevea brasiliensis
MALKLNPFLSQSHKLPSFALPPMASLRSPKFYMASTLKSGSKELENLKKPFMPPREVHVQVTHSMPPQKIEIFKSLENWAEENILIHLKPVEKCWQPQDFLPDPASDGFHEQVKELRERAKEIPDDYFVVLVGDMITEEALPTYQTMLNTLDGVRDETGASPTSWAIWTRAWTAEENRHGDLLNKYLYLSGRVDMRQIEKTIQYLIGSGMDPRTENSPYLGFIYTSFQERATFISHGNTARLAKEHGDIKLAQICGTIASDEKRHETAYTKIVEKLFEIDPDGTVMAFADMMRKKISMPAHLMYDGLDDNLFDHFSAVAQRLGVYTAKDYADILEFLVGRWKVDKLTGLSSEGQKAQDYVCRLPPRIRRLEERAQGRAKEATTIPFSWIFDREVKL

SEQ ID NO: 9 - protein, 396 aa, Jatropha curcas
MALKLNPFISQFHKLPTFALPPMANLRSPKFYMASTLKSGSKEVENLKKPFMPPREVHVQVTHSMPPQKIEIFKSLDEWAEQNILVHLKPVEKCWQPQDFLPDPSSDGFDEQVRELRERVKEIPDDYFVVLVGDMITEEALPTYQTMLNTLDGVRDETGASLTSWAIWTRAWTAEENRHGDLLNKYLYLSGRVDMRQIEKTIQYLIGSGMDPRTENSPYLGFIYTSFQERATFISHGNTARLAKEHGDIKLAQICGTIAADEKRHETAYTKIVEKLFEIDPDGTVLAFADMMRKKISMPAHLMYDGRDDNLFDHFSAVAQRLGVYTAKDYADILEFLVGRWKVDKLTGLSAEGQKAQDYVCRLPPRIRRLEERAQGRAKEGPTIPFSWIFDREVKL

SEQ ID NO: 10 - protein, 396 aa, Manihot esculenta
MALKLNPFLSQSQKLPSFALPPMASLRSPKFYMASTLKTGSKEVENLKKPFTPPREVHVQVTHSMPPQKIEIFKSLDDWAEKNILIHLKPVEKCWQPQDFLPDPASDGFDEQVKELRERAKEIPDDYLVVLVGDMITEEALPTYQTMLNTLDGVRDETGASLTSWAIWTRAWTAEENRHGDLLNKYLYLSGRVDMRQIEKTIQYLIGSGMDPRTENSPYLGFIYTSFQERATFISHGNTARLAKEHGDIKLAQICGTIAADEKRHETAYTKIVEKLFEIDPDGTVLAFADMMRKKISMPAHLMYDGRDDTLFDHFSAVAQRLGVYTAKDYADILEFLVGRWKVDKLTGLSSEGQEAQDYVCRLPPRIRRLEERAQGRAKEAATIPFSWIFDREVKL

SEQ ID NO: 11 - protein, 396 aa, Vernicia montana
MALKLNPFISQSQKFPSFALPPMANLRSPKFYMASTLRSGSKEIEHLKKPFMPPREVHVQVTHSMPSQKIEIFKSLEDWAEQNILVHLKPVEKCWQPQDFLPDPVSDGFDEQVKELRERAKEIPDDYFVVLVGDMITEEALPTYQTMLNTLDGVRDETGASLTSWAIWTRAWTAEENRHGDLLNKYLYLSGRVDMRQIEKTIQYLIGSGMDPRTENSPYLGFIYTSFQERATFISHGNTARHAKEHGDIKLAQICGTIASDEKRHETAYTKIVEKLFEIDPDGTVLAFADMMRKKISMPAHLMYDGRDDNLFDHFSAVAQRLGVYTAKDYADILEFLVGRWKVDKLTGLSAEGQKAQDYVCRLPPRIRRLEERAQGRAKEATTIPFSWIFEREVQL

SEQ ID NO: 12 - protein, 457 aa, Theobroma cacao
MHLISTKWALYLLSLSFAKCVFSLLWSKVKLLKQKPKENSQRGLKHQISREKNQNQANAEQMALKLNPITSQSQKLPYFALPPMASLRSPKFFMASTLRSGSKEVENVKKPFMPPREVHVQVTHSMPPQKIEIFKSLENWAEQNILVHLKPVEKCWQPQDFLPDPASDGFDEQVKELRERAKEIPDDYFVVLVGDMITEEALPTYQTMLNTLDGVLDETGASLTSWAIWTRAWTAEENRHGDLLNKYLYLSGRVDMRQIEKTIQYLIGSGMDPRTENSPYLGFIYTSFQERATFISHGNTARLAKEHGDFKLAQICGTIASDERRHETAYTKIVEKLFEIDPDGTVLAFADMMRKKISMPAHLMYDGRDDNLFDHFSAVAQRLGVYTAKDYADILEFLVERWKVKELTGLSADGRKAQDYVCGLPPRIRRLEERAQGRAKQAPSIPFSWIFDREVKL

SEQ ID NO: 13 - protein, 396 aa, Citrus clementina
MALKLSPFTTQTQKFPSFALPQMGSLRSPKFSMASTLRSNTKEVENLKKPFMPPREVHVQVTHSMPPQKIEIFKSMEDWAENNILVHLKPVEKCWQPQDFLPDPASDGFDEQVKELRERAKELPDDYFVVLVGDMITEEALPTYQTMLNTLDGVRDETGASLTSWAIWTRAWTAEENRHGDLLNKYLYLSGRVDMRQIEKTIQYLIGSGMDPRTENSPYLGFIYTSFQERATFISHGNTARLAKEHGDMKLAQICGTIASDEKRHETAYTKIVEKLFEIDPDGTIVSFADMMRKKISMPAHLMYDGRDDNLFEHFSAVAQRLGVYTAKDYADILEFLVGRWKVEKLTGLSGEGQKAQDYVCGLPARIRRLEERAQGRAKQGPTIPFSWIYDRQVQL

SEQ ID NO: 14 - DNA, 1239 bp, Ricinus communis
ttccggcaaataacaaaaaaccaaaagaaaaaggtaagaaaaaaaacaatggctctcaagctcaatcctttcctttctcaaacccaaaagttaccttctttcgctcttccaccaatggccagtaccagatctcctaagttctacatggcctctaccctcaagtctggttctaaggaagttgagaatctcaagaagcctttcatgcctcctcgggaggtacatgttcaggttacccattctatgccaccccaaaagattgagatctttaaatccctagacaattgggctgaggagaacattctggttcatctgaagccagttgagaaatgttggcaaccgcaggattttttgccagatcccgcctctgatggatttgatgagcaagtcagggaactcagggagagagcaaaggagattcctgatgattattttgttgttttggttggagacatgataacggaagaagcccttcccacttatcaaacaatgctgaataccttggatggagttcgggatgaaacaggtgcaagtcctacttcttgggcaatttggacaagggcatggactgcggaagagaatagacatggtgacctcctcaataagtatctctacctatctggacgagtggacatgaggcaaattgagaagacaattcaatatttgattggttcaggaatggatccacggacagaaaacagtccataccttgggttcatctatacatcattccaggaaagggcaaccttcatttctcatgggaacactgcccgacaagccaaagagcatggagacataaagttggctcaaatatgtggtacaattgctgcagatgagaagcgccatgagacagcctacacaaagatagtggaaaaactctttgagattgatcctgatggaactgttttggcttttgctgatatgatgagaaagaaaatttctatgcctgcacacttgatgtatgatggccgagatgataatctttttgaccacttttcagctgttgcgcagcgtcttggagtctacacagcaaaggattatgcagatatattggagttcttggtgggcagatggaaggtggataaactaacgggcctttcagctgagggacaaaaggctcaggactatgtttgtcggttacctccaagaattagaaggctggaagagagagctcaaggaagggcaaaggaagcacccaccatgcctttcagctggattttcgataggcaagtgaagctgtag

(19) United States
(12) Patent Application Publication
(10) Pub. No.: US 2021/0062163 A1
(43) Pub. Date: Mar. 4, 2021

(54) Stearoyl-ACP Desaturase and Variants Thereof Capable of Dioxygenase Chemistry and Converting Oleoyl-ACP to erythro-9,10-Dihydroxystearate

(71) Applicant: Brookhaven Science Associates, LLC, Upton, NY (US)

(72) Inventors: John Shanklin, Shoreham, NY (US); Edward J. Whittie, Greenport, NY (US)

(21) Appl. No.: 17/006,711

(22) Filed: Aug. 28, 2020

Related U.S. Application Data
(60) Provisional application No. 62/894,395, filed on Aug. 30, 2019.

(51) Int. Cl. C12N 9/02; C12P 7/64

(52) U.S. Cl. CPC C12N 9/0071; C12P 7/6409; C12Y 114/19002

(57) ABSTRACT

The invention provides wild type stearoyl-ACP type desaturase, and its mutants, particularly T117R and D280K, for converting oleoyl-ACP, the normal product of the stearoyl-ACP desaturase, to a vicinal diol, erythro 9, 10 dihydroxy stearate. The invention provides mutant or variant stearoyl-ACP type desaturase polypeptides having one or more amino acid substitutions, particularly one or more substitution at amino acid 117 and/or amino acid 280, of the plastid enzyme polypeptide. The mutant polypeptides provide for higher vicinal diol, particularly 9, 10 dihydroxy stearate, compared to wild type stearoyl-acyl carrier protein (ACP) desaturase, including when the mutant stearoyl-ACP type desaturase is expressed in host cells. Also provided are polynucleotides encoding the mutant stearoyl-ACP type desaturase, constructs and host cells comprising the polynucleotides, methods for producing a vicinal diol, erythro 9, 10 dihydroxy stearate, in host cells. The invention also relates to plants, particularly transgenic or recombinantly engineered plants, expressing one or more of the mutant a vicinal diol, erythro 9, 10 dihydroxy stearate polypeptides, as well as seeds derived from the plants.

### GOVERNMENT SUPPORT

This invention was made with Government support under contract number DE-SC0012704, awarded by the U.S. Department of Energy. The Government has certain rights in the invention.

### FIELD OF THE INVENTION

The present invention relates to wild type stearoyl-ACP type desaturase, and its mutants, particularly T117R and D280K, for converting oleoyl-ACP, the normal product of the stearoyl-ACP desaturase, to a vicinal diol, i.e., a saturated C18 fatty acid with hydroxy groups on adjacent C9 and C10 carbons, known as erythro 9, 10 dihydroxy stearate. This conversion may be useful for engineering of living systems to optimize the accumulation of vicinal diol fatty acid (VDFA).

### BACKGROUND OF THE INVENTION

Diiron clusters within the active sites of enzymes facilitate the binding of molecular oxygen and its derivatives and are able to perform redox chemistry which results in a range of chemical outcomes (Edmondson and Juynh, 1996). All diiron enzymes characterized to date belong to one of two separate classes, one soluble and the other membrane bound (Shanklin and Somerville, 1991). Both classes have the ability to catalyze the oxidation of unactivated C—H bonds to give a range of chemical outcomes (Shanklin and Cahoon, 1998; Fox et al., 2004). For instance, both soluble and membrane diiron enzyme classes contain desaturase enzymes that perform the stereo- and regioselective introduction of Z-(cis) double bonds into unactivated lipid acyl chains. The reactions are thought to proceed via a radical mechanism initiated by abstraction of a specific hydrogen from the substrate (Buist, 2004). Double bond formation may ensue via the abstraction of a second neighboring hydrogen. As predicted by Bloch (Bloch, 1969) and subsequently confirmed by X-ray crystallography (Lindqvist et al., 1996; Bai et al., 2015), the boomerang shape of the substrate binding channel within the desaturase drives the formation of the (Z)-olefinic fatty acids.

There is a diverse constellation of chemical outcomes performed by variant enzymes that are structurally related to the prototypical desaturase. The membrane bound diiron-containing plant fatty acid desaturase (FAD) family of FAD2 variant enzymes perform a variety of chemical transformations. Using oleate as a substrate, either desaturated or hydroxylated products may be obtained; using linoleate as a substrate, the corresponding epoxide, a conjugated double bond, or an acetylenic bond can be produced. Changes in chemoselectivity may be based on a relatively small number of amino acid sequence differences which presumably alter the relative orientation of the substrate with respect to the active site oxidant (Bhar et al., 2012). For instance, changes to four amino acid side chains was sufficient to predominantly convert a FAD2 into a hydroxylase and vice versa (Broun et al., 1998; Broadwater et al., 2002). Despite an increasing understanding of specificity determining residues within the FAD2-related diiron enzymes, there remains a need for further interpretation, which has been hindered by the lack of structural information for these enzymes. Publication of structures of several mammalian membrane-bound desaturase enzymes indicates that it may be possible to solve one of the plant FAD2 class at some point and it may be possible to correlate changes to the enzyme structure with distinct functional outcomes (Bai et al., 2015; Wang et al., 2015). Homology modeling may also be useful in elucidating mechanisms of enzymes such as FAD2 and FAD3 (Cai et al., 2018).

Vicinal diol fatty acids (VDFAs) refers to fatty acids with two hydroxyl groups on adjacent carbons and may have uses as specialty fatty acids. Such functionalization facilitates their use and application as activated feedstocks that can be chemically derivatized to form new compounds. VDFA have been identified in the oils of a number of plants including castor and Cardamine impatiens. While castor oil is abundant, the VDFA content is low in the approximate range of 1%. In contrast, VDFAs accumulate to approximately 25% in plants such as Cardamine impatiens, but Cardamine impatiens itself has limited or low seed yield and there may be other properties that render it less than suitable for agronomic production of oil. It remains desirable to create a large-scale supply of VDFA in for example crop plants, microbes, or other living systems.

This invention characterizes the capability of a stearoyl-ACP type desaturase to convert oleoyl-ACP to a vicinal diol, and is particularly directed to mutant or variant stearoyl-ACP type desaturases and their applicability to generate and increase vicinal diols and VDFAs and to provide a source of vicinal diols and VDFAs in seeds, plants and in other biological systems.

The citation of references herein shall not be construed as an admission that such is prior art to the present invention.

### SUMMARY OF THE INVENTION

The invention relates generally to methods and approaches for converting oleoyl-ACP to a vicinal diol. The invention provides a diiron enzyme, particularly a plant diiron enzyme, capable of a dioxygenase reaction mechanism to convert a double bond to a vicinal diol.

It has been recognized that wild type stearoyl-acyl carrier protein (ACP) type deasaturase enzyme, particularly natural or wild type stearoyl-ACP type deasaturase from castor (Ricinus communis) is capable of converting oleoyl-ACP to a vicinal diol, although vicinal diol is generated and accumulates at a low level, roughly 1% or somewhat less, on the order of 0.5%-1%, or about 0.7%, in castor oil. In accordance with the present invention, variant or mutant stearoyl-ACP type deasaturase polypeptides are provided wherein one or more amino acid substitution is introduced and wherein the variant or mutant desaturase is capable of converting oleoyl-ACP to a vicinal diol. In accordance with the invention, the variant or mutant plant stearoyl-ACP type deasaturase is capable of converting oleoyl-ACP to a vicinal diol, such that vicinal diol accumulates at an increased level, increasing by 10 fold or greater, such that at least 10%, up to 15%, up to 20%, up to 25%, up to 30% in plant seed oil. In accordance with the invention, the variant or mutant castor plant stearoyl-ACP type deasaturase is capable of converting oleoyl-ACP to a vicinal diol, such that vicinal diol accumulates at an increased level, increasing by 10 fold or greater, such that at least 10%, up to 15%, up to 20%, up to 25%, up to 30% in castor oil.

In an embodiment, the mutant or variant stearoyl-ACP desaturase is capable of accumulating a novel product erythro-9,10-dihydroxystearate.

The invention provides a mutant plant stearoyl-acyl carrier protein (ACP) desaturase polypeptide capable of catalyzing the conversion of oleoyl-ACP to erythro 9,10 dihydroxy stearate, wherein one or more amino acid is substituted and wherein the conversion of oleoyl-ACP to erythro 9,10 dihydroxy stearate is increased. In an embodiment, the conversion of oleoyl-ACP to erythro 9,10 dihydroxy stearate is increased by at least 10 fold compared the wild type or native, non mutant plant stearoyl-acyl carrier protein (ACP) desaturase polypeptide. In an embodiment, the mutant stearoyl-acyl carrier protein (ACP) desaturase polypeptide generates 9,10 dihydroxy stearate as a component of castor oil at or up to at least 10% of the total fatty acids. In an embodiment, the mutant stearoyl-acyl carrier protein (ACP) desaturase polypeptide generates 9,10 dihydroxy stearate as a component of castor oil at or up to at least 15% of the total fatty acids. In an embodiment, the mutant stearoyl-acyl carrier protein (ACP) desaturase polypeptide generates 9,10 dihydroxy stearate as a component of castor oil at or up to at least 20% of the total fatty acids. In an embodiment, the mutant stearoyl-acyl carrier protein (ACP) desaturase polypeptide generates 9,10 dihydroxy stearate as a component of castor oil at or up to at least 25% of the total fatty acids.

The invention provides a mutant plant diiron enzyme polypeptide capable of a dioxygenase reaction mechanism wherein a double bond is converted to a vicinal diol.

In an embodiment, the mutant plant diiron enzyme polypeptide is a mutant stearoyl-acyl carrier protein (ACP) desaturase polypeptide capable of catalyzing the conversion of oleoyl-ACP to erythro 9,10 dihydroxy stearate comprising:

(a) an amino acid replacement of the threonine (T) at amino acid residue 117 of the processed plastid polypeptide sequence and corresponding to residue 117 of SEQ ID NO: 2 or of the amino acid at the corresponding position in a plant stearoyl-ACP desaturase polypeptide;

(b) an amino acid replacement of the aspartic acid (D) at amino acid residue 280 of the processed plastid polypeptide sequence and corresponding to residue 280 of SEQ ID NO: 2 or of the amino acid at the corresponding position in a plant stearoyl-ACP desaturase polypeptide; or

(c) an amino acid replacement of the threonine (T) at amino acid residue 117 of the processed plastid polypeptide sequence and corresponding to residue 117 of SEQ ID NO: 2 or of the amino acid at the corresponding position in a plant stearoyl-ACP desaturase polypeptide and an amino acid replacement of the aspartic acid (D) at amino acid residue 280 of the processed plastid polypeptide sequence and corresponding to residue 280 of SEQ ID NO: 2 or of the amino acid at the corresponding position in a plant stearoyl-ACP desaturase polypeptide.

In an embodiment, the mutant plant diiron enzyme polypeptide is a mutant plant enzyme having at least 85% amino acid identity to the stearoyl-acyl carrier protein (ACP) desaturase polypeptide of SEQ ID NO:2 and is capable of a dioxygenase reaction mechanism wherein a double bond is converted to a vicinal diol or is capable of catalyzing the conversion of oleoyl-ACP to erythro 9,10 dihydroxy stearate comprising:

(a) an amino acid replacement of the threonine (T) at amino acid residue 117 of the processed plastid polypeptide sequence and corresponding to residue 117 of SEQ ID NO: 2 or of the amino acid at the corresponding position in a plant stearoyl-ACP desaturase polypeptide;

(b) an amino acid replacement of the aspartic acid (D) at amino acid residue 280 of the processed plastid polypeptide sequence and corresponding to residue 280 of SEQ ID NO: 2 or of the amino acid at the corresponding position in a plant stearoyl-ACP desaturase polypeptide; or

(c) an amino acid replacement of the threonine (T) at amino acid residue 117 of the processed plastid polypeptide sequence and corresponding to residue 117 of SEQ ID NO: 2 or of the amino acid at the corresponding position in a plant stearoyl-ACP desaturase polypeptide and an amino acid replacement of the aspartic acid (D) at amino acid residue 280 of the processed plastid polypeptide sequence and corresponding to residue 280 of SEQ ID NO: 2 or of the amino acid at the corresponding position in a plant stearoyl-ACP desaturase polypeptide.

In an embodiment, the mutant plant diiron enzyme polypeptide is a mutant plant enzyme having at least 90% amino acid identity to the stearoyl-acyl carrier protein (ACP) desaturase polypeptide of SEQ ID NO:2 and is capable of a dioxygenase reaction mechanism wherein a double bond is converted to a vicinal diol or is capable of catalyzing the conversion of oleoyl-ACP to erythro 9,10 dihydroxy stearate comprising:

(a) an amino acid replacement of the threonine (T) at amino acid residue 117 of the processed plastid polypeptide sequence and corresponding to residue 117 of SEQ ID NO: 2 or of the amino acid at the corresponding position in a plant stearoyl-ACP desaturase polypeptide;

(b) an amino acid replacement of the aspartic acid (D) at amino acid residue 280 of the processed plastid polypeptide sequence and corresponding to residue 280 of SEQ ID NO: 2 or of the amino acid at the corresponding position in a plant stearoyl-ACP desaturase polypeptide; or

(c) an amino acid replacement of the threonine (T) at amino acid residue 117 of the processed plastid polypeptide sequence and corresponding to residue 117 of SEQ ID NO: 2 or of the amino acid at the corresponding position in a plant stearoyl-ACP desaturase polypeptide and an amino acid replacement of the aspartic acid (D) at amino acid residue 280 of the processed plastid polypeptide sequence and corresponding to residue 280 of SEQ ID NO: 2 or of the amino acid at the corresponding position in a plant stearoyl-ACP desaturase polypeptide.

In an embodiment, the mutant plant enzyme polypeptide comprises an amino acid replacement at residue 117 or its corresponding position wherein the amino acid threonine or such other hydroxylic amino acid or amino acid having an uncharged polar R group is replaced with a basic amino acid or charged or nonpolar R group. In an embodiment, the mutant stearoyl-acyl carrier protein (ACP) desaturase polypeptide comprises an amino acid replacement at residue 117 or its corresponding position wherein the amino acid threonine or such other hydroxylic amino acid or amino acid having an uncharged polar R group is replaced with a basic amino acid or charged or nonpolar R group.

In an embodiment, the mutant plant enzyme polypeptide comprises an amino acid replacement at residue 280 or its corresponding position wherein the amino acid aspartic acid or such other acidic amino acid or amino acid having a polar R group is replaced with a basic amino acid or uncharged or nonpolar R group. In an embodiment, the mutant stearoyl-acyl carrier protein (ACP) desaturase polypeptide comprises an amino acid replacement at residue 280 or its corresponding position wherein the amino acid aspartic acid or such other acidic amino acid or amino acid having a polar R group is replaced with a basic amino acid or uncharged or nonpolar R group.

In an embodiment, the mutant plant enzyme polypeptide comprises an amino acid replacement at residue 117 or its corresponding position wherein the amino acid threonine or such other hydroxylic amino acid or amino acid having an uncharged polar R group is replaced with a basic amino acid or charged or nonpolar R group and further comprises an amino acid replacement at residue 280 or its corresponding position wherein the amino acid aspartic acid or such other acidic amino acid or amino acid having a polar R group is replaced with a basic amino acid or uncharged or nonpolar R group. In an embodiment, the mutant stearoyl-acyl carrier protein (ACP) desaturase polypeptide comprises an amino acid replacement at residue 117 or its corresponding position wherein the amino acid threonine or such other hydroxylic amino acid or amino acid having an uncharged polar R group is replaced with a basic amino acid or charged or nonpolar R group and further comprises an amino acid replacement at residue 280 or its corresponding position wherein the amino acid aspartic acid or such other acidic amino acid or amino acid having a polar R group is replaced with a basic amino acid or uncharged or nonpolar R group.

In an embodiment, the mutant plant enzyme polypeptide or the mutant stearoyl-acyl carrier protein (ACP) desaturase polypeptide comprises an amino acid replacement at residue 117 or its corresponding position wherein the amino acid threonine is replaced with a basic amino selected from arginine, lysine and histidine. In an embodiment, the mutant polypeptide comprises an amino acid replacement at residue 117 or its corresponding position wherein the amino acid threonine is replaced with an arginine.

In an embodiment, the mutant plant enzyme polypeptide or the mutant stearoyl-acyl carrier protein (ACP) desaturase polypeptide comprises an amino acid replacement at residue 280 or its corresponding position wherein the amino acid aspartic acid is replaced with a basic amino selected from arginine, lysine and histidine. In an embodiment, the mutant polypeptide comprises an amino acid replacement at residue 280 or its corresponding position wherein the amino acid threonine is replaced with a lysine.

In an embodiment, the mutant plant enzyme polypeptide catalyzes the conversion of substrate to a vicinal diol and generates at least 10 fold more vicinal diol than the wild type or native, non mutant plant enzyme polypeptide. In an embodiment, the mutant stearoyl-acyl carrier protein (ACP) desaturase polypeptide catalyzes the conversion of oleoyl-ACP to erythro 9,10 dihydroxy stearate and generates at least 10 fold more erythro 9,10 dihydroxy stearate than the wild type or native, non mutant plant stearoyl-acyl carrier protein (ACP) desaturase polypeptide.

In an embodiment, the mutant plant enzyme polypeptide catalyzes the conversion of substrate to a vicinal diol and generates at least 10 fold more vicinal diol than the wild type or native, non mutant plant enzyme polypeptide. In an embodiment, the mutant plant enzyme polypeptide catalyzes the conversion of substrate to a vicinal diol and generates at least 20 fold more vicinal diol than the wild type or native, non mutant plant enzyme polypeptide. In an embodiment, the mutant plant enzyme polypeptide catalyzes the conversion of substrate to a vicinal diol and generates at least 30 fold more vicinal diol than the wild type or native, non mutant plant enzyme polypeptide. In an embodiment, the mutant plant enzyme polypeptide catalyzes the conversion of substrate to a vicinal diol and generates at least 40 fold more vicinal diol than the wild type or native, non mutant plant enzyme polypeptide. In an embodiment, the mutant plant enzyme polypeptide catalyzes the conversion of substrate to a vicinal diol and generates at least 50 fold more vicinal diol than the wild type or native, non mutant plant enzyme polypeptide. the mutant plant enzyme polypeptide catalyzes the conversion of substrate to a vicinal diol and generates at least 10 fold, 20 fold, 30 fold, 40 fold, 50 fold, 60 fold, 80 fold, 100 fold more vicinal diol than the wild type or native, non mutant plant enzyme polypeptide. In an embodiment, the mutant stearoyl-acyl carrier protein (ACP) desaturase polypeptide catalyzes the conversion of oleoyl-ACP to erythro 9,10 dihydroxy stearate and generates at least 10 fold more erythro 9,10 dihydroxy stearate than the wild type or native, non mutant plant stearoyl-acyl carrier protein (ACP) desaturase polypeptide. In an embodiment, the mutant stearoyl-acyl carrier protein (ACP) desaturase polypeptide catalyzes the conversion of oleoyl-ACP to erythro 9,10 dihydroxy stearate and generates at least 20 fold more erythro 9,10 dihydroxy stearate than the wild type or native, non mutant plant stearoyl-acyl carrier protein (ACP) desaturase polypeptide.

In an embodiment, the mutant stearoyl-acyl carrier protein (ACP) desaturase polypeptide catalyzes the conversion of oleoyl-ACP to erythro 9,10 dihydroxy stearate and generates at least 30 fold more erythro 9,10 dihydroxy stearate than the wild type or native, non mutant plant stearoyl-acyl carrier protein (ACP) desaturase polypeptide. In an embodiment, the mutant stearoyl-acyl carrier protein (ACP) desaturase polypeptide catalyzes the conversion of oleoyl-ACP to erythro 9,10 dihydroxy stearate and generates at least 40 fold more erythro 9,10 dihydroxy stearate than the wild type or native, non mutant plant stearoyl-acyl carrier protein (ACP) desaturase polypeptide. In an embodiment, the mutant stearoyl-acyl carrier protein (ACP) desaturase polypeptide catalyzes the conversion of oleoyl-ACP to erythro 9,10 dihydroxy stearate and generates at least 50 fold more erythro 9,10 dihydroxy stearate than the wild type or native, non mutant plant stearoyl-acyl carrier protein (ACP) desaturase polypeptide. In an embodiment, the mutant stearoyl-acyl carrier protein (ACP) desaturase polypeptide catalyzes the conversion of oleoyl-ACP to erythro 9,10 dihydroxy stearate and generates at least 10 fold, 20 fold, 30 fold, 40 fold, 50 fold, 60 fold, 80 fold, 100 fold more erythro 9,10 dihydroxy stearate than the wild type or native, non mutant plant stearoyl-acyl carrier protein (ACP) desaturase polypeptide.

In an embodiment, the vicinal diol represents at least 10%, at least 20%, at least 30%, at least 40%, at least 50% of the product generated by the mutant enzyme polypeptide or the mutant stearoyl-acyl carrier protein (ACP) desaturase polypeptide. In an embodiment, the erythro 9,10 dihydroxy stearate represents at least 10%, at least 20%, at least 30%, at least 40%, at least 50% of the product generated by the mutant enzyme polypeptide or the mutant stearoyl-acyl carrier protein (ACP) desaturase polypeptide.

In an additional embodiment of the invention, the mutant plant enzyme polypeptide or the mutant stearoyl-acyl carrier protein (ACP) desaturase (s) can also be fused to a protein of interest, to form a fusion protein. The fusion protein (mutant stearoyl-acyl carrier protein (ACP) desaturase plus protein of interest) can be recombinantly expressed in a cell or organism or plant. In this the expressed fusion proteins can be used to purify and deliver the protein of interest, for a variety of applications. In a further aspect, the mutant plant enzyme polypeptide or the mutant stearoyl-acyl carrier protein (ACP) desaturase (s) may be labeled, including by attachment to a detectable or functional label.

Nucleic acids or polynucleotides encoding the mutant plant enzyme polypeptide and/or the mutant stearoyl-acyl carrier protein (ACP) desaturase polypeptides are also provided. The invention provides an isolated nucleic acid encoding the polypeptide, in particular the mutant stearoyl-acyl carrier protein (ACP) desaturase polypeptide. In an embodiment, nucleic acid is provided encoding one or more mutant plant enzyme polypeptide as described herein, including mutant plant enzyme polypeptide having one or more amino acid replacement or substitution as provided herein. In an embodiment, nucleic acid is provided encoding one or more mutant stearoyl-acyl carrier protein (ACP) desaturase as described herein, including mutant stearoyl-acyl carrier protein (ACP) desaturase having one or more amino acid replacement or substitution as provided herein. In an embodiment, nucleic acid is provided encoding one or more mutant plant enzyme polypeptide and/or one or more mutant stearoyl-acyl carrier protein (ACP) desaturase as described herein, including mutant plant enzyme polypeptide or mutant stearoyl-acyl carrier protein (ACP) desaturase having a replacement at plastid enzyme amino acid 117 or the corresponding position thereof as provided herein. In an embodiment, nucleic acid is provided encoding one or more mutant plant enzyme polypeptide or mutant stearoyl-acyl carrier protein (ACP) desaturase as described herein, including mutant plant enzyme polypeptide or mutant stearoyl-acyl carrier protein (ACP) desaturase having a replacement at plastid enzyme amino acid 280 or the corresponding position thereof as provided herein.

In a further embodiment the polynucleotide encodes a fusion protein including the modified stearoyl-acyl carrier protein (ACP) desaturase fused to a protein of interest.

In a further aspect the invention provides an expression construct comprising a polynucleotide of the invention. In one embodiment the polynucleotide in the construct is operably linked to a promoter sequence. In one embodiment the promoter sequence is capable of driving expression of the polynucleotide in a vegetative tissue of a plant. In another embodiment the promoter sequence is capable of driving expression of the polynucleotide in a seed of a plant. In a further embodiment the promoter sequence is capable of driving expression of the polynucleotide in the pollen of a plant. In a further embodiment the promoter sequence is capable of driving expression of the polynucleotide in a bacterial cell or yeast cell.

In an embodiment, the invention includes a recombinant vector comprising the nucleic acid of the invention. In another aspect, the invention provides a construct containing a polynucleotide that encodes a mutant plant enzyme polypeptide or mutant stearoyl-acyl carrier protein (ACP) desaturase as provided herein. In various embodiments, the construct can be linked to a promoter sequence capable of driving its expression in various host cells. As such, the invention also provides use of the constructs to induce a host cell to express a modified or mutant plant enzyme polypeptide or a modified or mutant stearoyl-acyl carrier protein (ACP) desaturase. In yet another embodiment the construct is located in an appropriate position and orientation of a suitable functional endogenous promoter such that the expression of the construct occurs. In various embodiments, the construct can be expressed in a bacterial, plant, fungal or algal cell. In one embodiment where the construct is expressed in a plant cell, the cell may be of vegetative, seed, pollen or fruit tissue.

In another aspect the invention provides a host cell comprising a construct and mutant plant enzyme polypeptide or mutant stearoyl-acyl carrier protein (ACP) desaturase of the invention. In an aspect the invention provides a host cell genetically modified to comprise a polynucleotide of the invention. In a further aspect the invention provides a host cell genetically modified to express a polynucleotide of the invention. In a further aspect the invention provides a host cell genetically modified to express a polypeptide of the invention. In a further embodiment, host cell(s) comprising the vector are provided. A host cell and host cells recombinantly engineered to heterologously produce the mutant plant enzyme polypeptide or mutant stearoyl-acyl carrier protein (ACP) desaturase polypeptide are provided herein. In embodiments, host cell(s) are recombinantly engineered to produce mutant plant enzyme polypeptide or mutant stearoyl-acyl carrier protein (ACP) desaturase by introducing nucleic acid encoding the mutant polypeptide.

In an embodiment, the host cell is a plant cell. In an embodiment, the host cell is a bacterial cell. In an embodiment, the host cell is a plant cell, bacterial cell or yeast cell or fungi.

In a further embodiment the nucleic acid is operably linked to a promoter sequence. The promoter sequence may capable of driving expression of the nucleic acid sequence in a vegetative tissue of a plant. In one aspect the promoter sequence is capable of driving expression of the nucleic acid sequence in a seed of a plant or in the pollen of a plant. The promoter sequence may be capable of driving expression of the polynucleotide in a bacterial cell or in a yeast cell.

In an embodiment of the invention, a host plant comprising a vector encoding the mutant plant enzyme polypeptide or mutant stearoyl-acyl carrier protein (ACP) desaturase polypeptide or recombinantly engineered to heterologously produce the polypeptide is provided herein. The host plant may be recombinantly engineered to overproduce the mutant plant enzyme polypeptide or mutant stearoyl-acyl carrier protein (ACP) desaturase polypeptide. In an embodiment, the plant is a castor plant or other seed oil plant. Suitable seed oil plants are known and available to one skilled in the art, including as described herein. In an embodiment, a seed oil plant is selected that is capable of being genetically engineered and recombinantly manipulated to produce or overproduce the mutant polypeptide.

The invention provides a genetically modified eukaryotic host cell which is genetically modified with a nucleic acid encoding a mutant plant enzyme polypeptide or mutant stearoyl-acyl carrier protein (ACP) desaturase polypeptide as provided herein. In an embodiment, the host cell produces vicinal diol. In an embodiment, the host cell produces erythro 9,10 dihydroxy stearate.

The host cell may be any suitable type of cell, including a prokaryotic cell or a eukaryotic cell. In one embodiment the host cell is selected from a bacterial cell, a yeast cell, a fungal cell, an insect cell, algal cell, and a plant cell. In a particular embodiment the host cell is a plant cell. The host cell may be a suitable bacterial cell, yeast cell, fungal cell, an animal cell or a plant cell. In a particular embodiment, the host cell is a bacterial cell.

The invention includes methods for producing a vicinal diol fatty acid in a host cell, the method comprising: a) introducing into a host cell at least one nucleic acid encoding a mutant plant enzyme polypeptide or mutant stearoyl-acyl carrier protein (ACP) desaturase as provided herein or otherwise engineering the host cell to produce a mutant plant enzyme polypeptide or mutant stearoyl-acyl carrier protein (ACP) desaturase as described and provided herein; and b) culturing the host cell in order to express the mutant plant enzyme polypeptide or mutant stearoyl-acyl carrier protein (ACP) desaturase. The invention includes methods for producing a vicinal diol fatty acid in a host cell, the method comprising: a) introducing into a host cell at least one nucleic acid encoding a mutant stearoyl-acyl carrier protein (ACP) desaturase as provided herein or otherwise engineering the host cell to produce a mutant stearoyl-acyl carrier protein (ACP) desaturase as described and provided herein; and b) culturing the host cell in order to express the mutant stearoyl-acyl carrier protein (ACP) desaturase.

In a further embodiment, methods are provided for producing vicinal diol or erythro 9,10 dihydroxy stearate in a host cell, the method comprising: a) introducing into a host cell at least one nucleic acid encoding a mutant plant enzyme polypeptide or mutant stearoyl-acyl carrier protein (ACP) desaturase as provided herein or otherwise engineering the host cell to produce a mutant plant enzyme polypeptide or mutant stearoyl-acyl carrier protein (ACP) desaturase hereof, and introducing a substrate for the enzyme polypeptide or the stearoyl-acyl carrier protein (ACP) desaturase enzyme; and b) culturing the host cell in order to express the modified or mutant plant enzyme polypeptide or modified or mutant stearoyl-acyl carrier protein (ACP) desaturase, whereby the substrate is converted to a vicinal diol or to erythro 9,10 dihydroxy stearate. In a further embodiment, methods are provided for producing erythro 9,10 dihydroxy stearate in a host cell, the method comprising: a) introducing into a host cell at least one nucleic acid encoding a mutant stearoyl-acyl carrier protein (ACP) desaturase as provided herein or otherwise engineering the host cell to produce a mutant stearoyl-acyl carrier protein (ACP) desaturase hereof, and introducing a substrate for the stearoyl-acyl carrier protein (ACP) desaturase enzyme; and b) culturing the host cell in order to express the modified or mutant stearoyl-acyl carrier protein (ACP) desaturase, whereby the substrate is converted to erythro 9,10 dihydroxy stearate.

The invention further provides a plant comprising a plant cell of the invention. In one aspect the invention provides a plant comprising a construct of the invention. In an aspect the invention provides a plant genetically modified to comprise or to express a polynucleotide of the invention. In an aspect the invention provides a plant genetically modified to comprise or to express a polypeptide of the invention. In a further embodiment the plant expresses a mutant plant enzyme polypeptide or a mutant stearoyl-acyl carrier protein (ACP) desaturase provided herein and encoded by the polynucleotide or nucleic acid of the invention. In a further embodiment the plant expresses a mutant stearoyl-acyl carrier protein (ACP) desaturase provided herein and encoded by the polynucleotide or nucleic acid of the invention.

The nucleic acid or polynucleotide of the invention may be operably linked to a promoter sequence. In an aspect, the promoter is suitable and applicable for expression in plants. In an aspect, the promoter is a constitutive promoter. In an aspect, the promoter is an inducible promoter. In an aspect, the promoter is a plant specific promoter, or a promoter directing expression in leaves, tissues or seeds of a plant. In an aspect, the promoter sequence is capable of driving expression of the nucleic acid sequence in a vegetative tissue of a plant. In one embodiment the promoter sequence is capable of driving expression of the nucleic acid sequence in a seed of a plant. In one embodiment the promoter sequence is capable of driving expression of the nucleic acid sequence in the pollen of a plant. In aspects, the promoter may be the constitutive promoter 35S or may be a seed promoter, particularly a strong seed promoter such as the promoter for the gene phaseolin.

In a further aspect the invention provides a composition comprising a mutant plant enzyme polypeptide or mutant stearoyl-acyl carrier protein (ACP) desaturase of the invention. In one embodiment the composition comprises the mutant plant enzyme polypeptide or mutant stearoyl-acyl carrier protein (ACP) desaturase and a suitable carrier.

The mutant plant enzyme polypeptide(s) may be modified naturally occurring plant enzyme polypeptide(s). The mutant stearoyl-acyl carrier protein (ACP) desaturase(s) may be modified naturally occurring stearoyl-acyl carrier protein (ACP) desaturase(s). The plants from which the un-modified or naturally occurring plant enzyme polypeptide sequences are derived may be from any plant species that contains a polypeptide enzyme having at least 85% amino acid identity or at least 90% amino acid identity to the castor stearoyl-acyl carrier protein (ACP) desaturase and polynucleotide sequences encoding a polypeptide enzyme having at least 85% amino acid identity or at least 90% amino acid identity to the castor stearoyl-acyl carrier protein (ACP) desaturase. The plant cells in which the mutant plant enzyme polypeptide(s) are expressed may be from any plant species. The plants from which the un-modified or naturally occurring plant enzyme polypeptide or stearoyl-acyl carrier protein (ACP) desaturase sequences are derived may be from any plant species that contains stearoyl-acyl carrier protein (ACP) desaturase and polynucleotide sequences encoding stearoyl-acyl carrier protein (ACP) desaturase. The plant cells in which the mutant stearoyl-acyl carrier protein (ACP) desaturase(s) are expressed may be from any plant species. The plants in which the mutant stearoyl-acyl carrier protein (ACP) desaturase are expressed may be from any plant species. In one embodiment the plant cell or plant, is derived from a gymnosperm plant species. In a further embodiment the plant cell or plant, is derived from an angiosperm plant species. In a further embodiment the plant cell or plant, is derived from a from dicotyledonous plant species. In a further embodiment the plant cell or plant, is derived from a monocotyledonous plant species. The plant or plant cell may be seed oil producing plant. The plant or plant cell may be a castor plant cell.

In one embodiment the plant accumulates more vicinal diol in its non-photosynthetic tissues/organs than does a control plant. In a further embodiment the plant accumulates at least 10%, more preferably at least 15%, more preferably at least 20%, more preferably at least 25%, more preferably at least 30%, more preferably at least 40%, more preferably at least 50%, more preferably at least 60%, more preferably at least 80%, more preferably at least 100% more vicinal diol in its non-photosynthetic tissues/organs than does a control plant.

In one embodiment the plant accumulates more 9,10 dihydroxy stearate in its non-photosynthetic tissues/organs than does a control plant. In a further embodiment the plant accumulates at least 10%, more preferably at least 15%, more preferably at least 20%, more preferably at least 25%, more preferably at least 30%, more preferably at least 40%, more preferably at least 50%, more preferably at least 60%, more preferably at least 80%, more preferably at least 100% more 9,10 dihydroxy stearate in its non-photosynthetic tissues/organs than does a control plant.

In embodiments of all aspects of the invention, the mutant plant enzyme polypeptide may be an acyl-Co-A integral membrane desaturase enzyme polypeptide. In embodiments of all aspects of the invention, the mutant plant enzyme polypeptide may be an acyl-Co-A integral membrane desaturase enzyme polypeptide, and may be an Arabidopsis stearoyl ACP desaturase sequence.

Suitable control plants include non-transformed or wild-type versions of plant of the same variety and/or species as the transformed plant used in the method of the invention. Suitable control plants also include plants of the same variety and or species as the transformed plant that are transformed with a control construct. Suitable control plants also include plants that have not been transformed with a polynucleotide encoding a mutant stearoyl-acyl carrier protein (ACP) desaturase provided herein. Suitable control plants also include plants that do not express a mutant stearoyl-acyl carrier protein (ACP) desaturase provided herein.

Other objects and advantages will become apparent to those skilled in the art from a review of the following description which proceeds with reference to the following illustrative drawings.

### BRIEF DESCRIPTION OF THE DRAWINGS

FIG. 1 provides chromatograms and GC-MS elution profiles of TMS derivatives, particularly of 18:1-ACP substrate (A) and product distributions for the castor desaturase triple mutant T117R G188L D280K (B), and each of single mutants T117R (C), G188L (D), and D280K (E) reveals a novel fatty acid species labeled as peak 5. Product profile of wild type castor desaturase is included (F) as a control. Peak identities: Z18:1Δ9 (1); Z18:1Δ11 (2); Z18:1Δ10 9OH (3); E18:1Δ10 9OH (4).

FIG. 2 depicts the novel fatty acid product is 9, 10-dihydroxystearate. Comparison of mass spectra of TMS derivatives of the novel enzymatic product produced by the castor desaturase T117R mutant (A) and an authentic erythro 9,10 dihydroxy stearate standard (C) and the fragmentation pattern giving rise to the major ions at 215 and 259 AMU (B).

FIG. 3 depicts that the 9,10-dihydroxystearate produced by the castor T117R mutant is solely in the erythro configuration. Gas chromatograms of 9,10-dihydroxy-stearates are compared for the reaction product of T117R (A) to those of standards: threo configuration (B), the erythro configuration (C), a mixture of the T117R product and the threo standard (D), and the T117R product and the erythro standard (E).

FIG. 4 depicts two potential schemes for the conversion of oleate to erythro 9,10 dihydroxystearate by a diiron-containing desaturase-dioxygenase. The initial bridged hydroperoxo species in both mechanisms is inspired by large-scale multireference ab initio calculations on a related enzyme (Chalupsky et al, 2014).

FIG. 5 depicts that both hydroxyl oxygens of 9,10-dihydroxystearate are derived from molecular oxygen. TMS derivatives of 9, 10-dihydroxystearate product from the castor desaturase T117R mutant using 18:1 11-d2-substrate under air (A), or ¹⁸O₂ shown in the diagram as O* (B).

FIG. 6 depicts that 9,10 dihydroxy stearate formation is the result of a single dioxygenase reaction. Chromatograms and corresponding mass spectra of acetonide derivatives of 9,10 dihydroxy stearate from reactions carried out under ¹⁶O₂ (A), equimolar ¹⁶O₂ and ¹⁸O₂ (B), and ¹⁸O₂ (C). Also depicted is an authentic erythro 9,10 dihydroxy stearate standard (D) along with a diagram of its fragmentation (E).

FIG. 7 depicts that upon prolonged incubation, the castor wild-type desaturase can convert 18:1 substrate to erythro-9, 10-dihydroxystearate. Peak identities: Z18:1Δ9 (1); Z18:1Δ11 (2); Z18:1Δ10 9OH (3); E18:1Δ10 9OH (4); 9,10-dihydroxystearate (5).

FIG. 8 depicts that low levels of erythro-9, 10-dihydroxystearate are present in developing castor embryos. Gas chromatogram of TMS derivatives of castor embryos (A) and the mass spectrum corresponding to 8, i.e., 9,10-dihydroxystearate (B). Peak identities: 16:0 (1), 18:0 (2), 18:1Δ9 (3), 18:1Δ11 (4), 18:2Δ9,12 (5), 12-OH 18:1Δ9 (6), 18:3Δ9,12,15 (7), 9, 10 OH 18:0 (8). C to E, GC peaks for TMS derivative of 9,10 dihydroxystearate from castor embryo (C), 9,10-dihydroxystearate from castor developing embryos mixed with authentic threo-9,10-dihydroxystearate standard (D), and authentic erythro-9,10-dihydroxystearate standard (E).

FIG. 9 provides the structural relationships of compounds discussed herein. Shown are 1 Stearoyl ACP, showing two hydrogens at C-9,10 that are removed by desaturase; 2 Oleoyl ACP, the product of a stearoyl 9,10 desaturation; 3 Erythro-9(R), 10 (R)-dihydroxystearoyl ACP, the predicted product of a one-step direct oleate dihydroxylation; and 4 Threo-9(S), 10 (R)-dihydroxystearoyl ACP, a possible product of an enzymatic two-step oleate epoxidation/hydrolysis sequence.

FIG. 10 provides a comparison of the stearoyl-ACP desaturase amino acid sequence of castor (Ricinus communis) starting with methionine (SEQ ID NO: 7) with various other plant stearoyl-ACP desaturase enzyme sequences. The castor enzyme T117, G188 and D280 amino acids are shown in bold and underlined. Amino acids in other plants that are distinct or variant from the R. communis castor enzyme sequence are shown in bold and any R. communis amino acid which is altered or variant (either conservatively or non-conservatively) in another of the plant sequences depicted is designated by an asterisk (*) underneath the applicable amino acid. The plant stearoyl-ACP desaturase sequences are as follows: Hevea brasiliensis, stearoyl-(acyl-carrier-protein) 9-desaturase, chloroplastic (XP_021688869.1) (SEQ ID NO:8); Jatropha curcas, acyl-(acyl-carrier-protein) desaturase, Chloroplastic (NP_001292942.1) (SEQ ID NO:9); Manihot esculenta, stearoyl-(acyl-carrier-protein) 9-desaturase, chloroplastic (XP_021610569) (SEQ ID NO:10); Vernicia montana, stearoyl-ACP desaturase (ABU50334.1) (SEQ ID NO:11); Theobroma cacao, Plant stearoyl-acyl-carrier-protein desaturase family protein (EOY04657.1) (SEQ ID NO:12); Citrus clementina, stearoyl-(acyl-carrier-protein) 9-desaturase, chloroplastic (XP_006442769.1) (SEQ ID NO:13).

### DETAILED DESCRIPTION

In accordance with the present invention there may be employed conventional molecular biology, microbiology, and recombinant DNA techniques within the skill of the art.

Therefore, if appearing herein, the following terms shall have the definitions set out below.

The amino acid residues described herein are preferred to be in the “L” isomeric form. However, residues in the “D” isomeric form can be substituted for any L-amino acid residue, as long as the desired functional property of immunoglobulin-binding is retained by the polypeptide. NH₂ refers to the free amino group present at the amino terminus of a polypeptide. COOH refers to the free carboxy group present at the carboxy terminus of a polypeptide. In keeping with standard polypeptide nomenclature, J. Biol. Chem., 243:3552-59 (1969), abbreviations for amino acid residues are shown in the following Table of Correspondence:

TABLE OF CORRESPONDENCE

| SYMBOL | | |
|---|---|---|
| 1-Letter | 3-Letter | AMINO ACID |
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The above Table is presented to correlate the three-letter and one-letter notations which may appear alternately herein.

Amino acids may be grouped according to the characteristics of their side chains, for example:

Aliphatic—alanine, glycine, isoleucine, leucine, proline, valine
Aromatic—phenylalanine, tryptophan, tyrosine
Acidic—aspartic acid, glutamic acid
Basic—arginine, histidine, lysine
Hydroxylic—serine, threonine
Sulphur-containing—cysteine, methionine
Amidic (containing amide group)—asparagine, glutamine

Mutants of the polypeptide of the present invention contemplate amino acid substitutions or replacements wherein one type of amino acid is replaced or substituted with a distinct amino acid—in terms of size, side chain character, charge etc—wherein the substitution results in altered function, activity or substrate—product relationship of the mutant polypeptide. Not all amino acid replacements have the same effect on function or structure of protein. The magnitude of this process may vary depending on how similar or dissimilar the replaced amino acids are, as well as on their position in the sequence or the structure. Similarity between amino acids can be calculated based on substitution matrices, physico-chemical distance, or simple properties such as amino acid size or charge (see also amino acid chemical properties). Usually amino acids are thus classified into two types—conservative and non-conservative substitutions or replacements. Conservative substitution or replacement—an amino acid is exchanged with another that has similar properties, such as similar biochemical properties (e.g. charge, hydrophobicity and size). This type of replacement is generally expected to not result in dysfunction or change in function in the corresponding protein. Non-conservative substitution or replacement—an amino acid is exchanged into another with different properties. This can lead to changes in protein structure or function, which can cause potentially lead to changes in activity or in phenotype, sometimes pathogenic.

The following is one example of various groupings of amino acids:

Amino acids with nonpolar R groups: Alanine, Valine, Leucine, Isoleucine, Proline, Phenylalanine, Tryptophan, Methionine
Amino acids with uncharged polar R groups: Glycine, Serine, Threonine, Cysteine, Tyrosine, Asparagine, Glutamine
Amino acids with charged polar R groups (negatively charged at pH 6.0): Aspartic acid, Glutamic acid
Basic amino acids (positively charged at pH 6.0): Lysine, Arginine, Histidine
Another grouping may be those amino acids with phenyl groups: Phenylalanine, Tryptophan

### Tyrosine

Particularly preferred conserved substitutions include:

Lys for Arg and vice versa such that a positive charge may be maintained;

Glu for Asp and vice versa such that a negative charge may be maintained;

Ser for Thr such that a free —OH can be maintained; and

Gln for Asn such that a free NH₂ can be maintained.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide,” as used herein in referring to the probe of the present invention, is defined as a molecule comprised of two or more ribonucleotides, preferably more than three. Its exact size will depend upon many factors which, in turn, depend upon the ultimate function and use of the oligonucleotide.

The term “probe” refers to a short polynucleotide that is used to detect a polynucleotide sequence that is complementary to the probe, in a hybridization-based assay. The probe may consist of a “fragment” of a polynucleotide as defined herein.

The term “primer” as used herein refers to a short polynucleotide, usually having a free 3′OH group, that is hybridized to a template and used for priming polymerization of a polynucleotide complementary to the target. A “primer” may be an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra. It should be appreciated that also within the scope of the present invention are DNA sequences encoding which code for a having the same amino acid sequence as SEQ ID NO:, but which are degenerate to SEQ ID NO:. By “degenerate to” is meant that a different three-letter codon is used to specify a particular amino acid. It is well known in the art that the following codons can be used interchangeably to code for each specific amino acid:

| | |
|---|---|
| Phenylalanine (Phe or F) | UUU or UUC |
| Leucine (Leu or L) | UUA or UUG or CUU or CUC or CUA or |
| | CUG |
| Isoleucine (Ile or I) | AUU or AUC or AUA |
| Methionine (Met or M) | AUG |
| Valine (Val or V) | GUU or GUC of GUA or GUG |
| Serine (Ser or S) | UCU or UCC or UCA or UCG or AGU or |
| | AGC |
| Proline (Pro or P) | CCU or CCC or CCA or CCG |
| Threonine (Thr or T) | ACU or ACC or ACA or ACG |
| Alanine (Ala or A) | GCU or GCG or GCA or GCG |
| Tyrosine (Tyr or Y) | UAU or UAC |
| Histidine (His or H) | CAU or CAC |
| Glutamine (Gln or Q) | CAA or CAG |
| Asparagine (Asn or N) | AAU or AAC |
| Lysine (Lys or K) | AAA or AAG |
| Aspartic Acid (Asp or D) | GAU or GAC |
| Glutamic Acid (Glu or E) | GAA or GAG |
| Cysteine (Cys or C) | UGU or UGC |
| Arginine (Arg or R) | CGU or CGC or CGA or CGG or AGA or |
| | AGG |
| Glycine (Gly or G) | GGU or GGC or GGA or GGG |
| Tryptophan (Trp or W) | UGG |
| Termination codon | UAA (ochre) or UAG (amber) or UGA (opal) |

It should be understood that the codons specified above are for RNA sequences. The corresponding codons for DNA have a T substituted for U.

Mutations can be made in stearoyl-ACP desaturase sequence(s) including in SEQ ID NO:1, 2 or 7 as provided herein such that a particular codon is changed to a codon which codes for a different amino acid. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping).

Two amino acid sequences are “substantially homologous” when at least about 70% of the amino acid residues (preferably at least about 80%, and most preferably at least about 90 or 95%) are identical, or represent conservative substitutions.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20^{N}C below the predicted or determined Tₘ with washes of higher stringency, if desired.

The term “polynucleotide(s),” as used herein, means a single or double-stranded deoxyribonucleotide or ribonucleotide polymer of any length but preferably at least 15 nucleotides, and include as non-limiting examples, coding and non-coding sequences of a gene, sense and antisense sequences complements, exons, introns, genomic DNA, cDNA, pre-mRNA, mRNA, rRNA, siRNA, miRNA, tRNA, ribozymes, recombinant polypeptides, isolated and purified naturally occurring DNA or RNA sequences, synthetic RNA and DNA sequences, nucleic acid probes, primers and fragments.

A “fragment” of a polynucleotide sequence provided herein is a subsequence of contiguous nucleotides that is capable of specific hybridization to a target of interest, e.g., a sequence that is at least 15 nucleotides in length. The fragments of the invention comprise 15 nucleotides, preferably at least 20 nucleotides, more preferably at least 25 nucleotides, more preferably at least 30 nucleotides, more preferably at least 35 nucleotides, more preferably at least 40 nucleotides, more preferably at least 45 nucleotides, more preferably at least 50 nucleotides, more preferably at least 60 nucleotides, more preferably at least 70 nucleotides, more preferably at least 80 nucleotides, more preferably at least 90 nucleotides, more preferably at least 100 nucleotides, more preferably at least 150 nucleotides, more preferably at least 200 nucleotides, more preferably at least 250 nucleotides, more preferably at least 300 nucleotides, more preferably at least 350 nucleotides, more preferably at least 400 nucleotides, more preferably at least 450 nucleotides and most preferably at least 500 nucleotides of contiguous nucleotides of a polynucleotide disclosed. A fragment of a polynucleotide sequence can be used in antisense, RNA interference (RNAi), gene silencing, triple helix or ribozyme technology, or as a primer, a probe, included in a microarray, or used in polynucleotide-based selection methods of the invention.

The term “polypeptide”, as used herein, encompasses amino acid chains of any length but preferably at least 5 amino acids, including full-length proteins, in which amino acid residues are linked by covalent peptide bonds. Polypeptides of the present invention, or used in the methods of the invention, may be purified natural products, or may be produced partially or wholly using recombinant or synthetic techniques. The term may refer to a polypeptide, an aggregate of a polypeptide such as a dimer or other multimer, a fusion polypeptide, a polypeptide fragment, a polypeptide variant, or derivative thereof.

A “fragment” of a polypeptide is a subsequence of the polypeptide that performs a function that is required for the biological activity and/or provides three dimensional structure of the polypeptide. The term may refer to a polypeptide, an aggregate of a polypeptide such as a dimer or other multimer, a fusion polypeptide, a polypeptide fragment, a polypeptide variant, or derivative thereof capable of performing the above enzymatic activity.

The term “isolated” as applied to the polynucleotide or polypeptide sequences disclosed herein is used to refer to sequences that are removed from their natural cellular environment. An isolated molecule may be obtained by any method or combination of methods including biochemical, recombinant, and synthetic techniques.

The term “recombinant” refers to a polynucleotide sequence that is removed from sequences that surround it in its natural context and/or is recombined with sequences that are not present in its natural context.

A “recombinant” polypeptide sequence is produced by translation from a “recombinant” polynucleotide sequence.

The term “derived from” with respect to polynucleotides or polypeptides of the invention being derived from a particular genera or species, means that the polynucleotide or polypeptide has the same sequence as a polynucleotide or polypeptide found naturally in that genera or species. The polynucleotide or polypeptide, derived from a particular genera or species, may therefore be produced synthetically or recombinantly.

As used herein, the term “variant” refers to polynucleotide or polypeptide sequences different from the specifically identified sequences, wherein one or more nucleotides or amino acid residues is deleted, substituted, or added, in a particular aspect wherein one or more nucleotides or amino acid residues is substituted. Variants may be naturally occurring allelic variants, or non-naturally occurring variants. Variants may be from the same or from other species and may encompass homologues, paralogues and orthologues. In certain embodiments, variants of the inventive polypeptides and polypeptides possess biological activities that are the same or similar to those of the inventive polypeptides or polypeptides. The term “variant” with reference to polypeptides and polynucleotides encompasses all forms of polypeptides and polynucleotides as defined herein.

Polynucleotide and polypeptide sequence identity can be determined in the following manner. The subject polynucleotide or polypeptide sequence is compared to a candidate polynucleotide or polypeptide sequence using BLASTN or BLASTP (from the BLAST suite of programs, version 2.2.5 [November 2002]) in bl2seq (Tatiana A. Tatusova, Thomas L. Madden (1999), “Blast 2 sequences—a new tool for comparing protein and nucleotide sequences”, FEMS Microbiol Lett. 174:247-250), which is publicly available from NCBI (ftp://ftp.ncbi.nih.gov/blast/).

Polynucleotide and polypeptide variants of the present invention also encompass those which exhibit a similarity to one or more of the specifically identified sequences that is likely to preserve the functional equivalence of those sequences and which could not reasonably be expected to have occurred by random chance. Such sequence similarity with respect to polypeptides and polynucleotides may be determined using the publicly available bl2seq program from the BLAST suite of programs (version 2.2.5 [November 2002]) from NCBI (ftp://ftp.ncbi.nih.gov/blast/).

The term “hybridize under stringent conditions”, and grammatical equivalents thereof, refers to the ability of a polynucleotide molecule to hybridize to a target polynucleotide molecule (such as a target polynucleotide molecule immobilized on a DNA or RNA blot, such as a Southern blot or Northern blot) under defined conditions of temperature and salt concentration. The ability to hybridize under stringent hybridization conditions can be determined by initially hybridizing under less stringent conditions then increasing the stringency to the desired stringency.

The term “variant” with reference to polypeptides encompasses naturally occurring, recombinantly and synthetically produced polypeptides. Variant polypeptide sequences preferably exhibit at least 50%, more preferably at least 60%, more preferably at least 70%, more preferably at least 75%, more preferably at least 80%, more preferably at least 85%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a sequences of the present invention. Identity is found over a comparison window of at least 20 amino acid positions, preferably at least 50 amino acid positions, more preferably at least 100 amino acid positions, and most preferably over the entire length of a polypeptide of the invention. Polypeptide sequence identity can be determined in the following manner. The subject polypeptide sequence is compared to a candidate polypeptide sequence using BLASTP (from the BLAST suite of programs, version 2.2.5 [November 2002]) in bl2seq, which is publicly available from NCBI (ncbi.nih.gov/blast).

Polypeptide variants of the present invention, or used in the methods of the invention, also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available bl2seq program from the BLAST suite of programs (version 2.2.5 [November 2002]) from NCBI (ftp://ftp.ncbi.nih.gov/blast/).

Conservative substitutions of one or several amino acids of a described polypeptide sequence without significantly altering its biological activity are also included in the invention. A skilled artisan will be aware of methods for making phenotypically silent amino acid substitutions (see, e.g., Bowie et al., 1990, Science 247, 1306).

The term “genetic construct” refers to a polynucleotide molecule, usually double-stranded DNA, which may have inserted into it another polynucleotide molecule (the insert polynucleotide molecule) such as, but not limited to, a cDNA molecule. A genetic construct may contain the necessary elements that permit transcribing the insert polynucleotide molecule, and, optionally, translating the transcript into a polypeptide. The insert polynucleotide molecule may be derived from the host cell, or may be derived from a different cell or organism and/or may be a recombinant polynucleotide. Once inside the host cell the genetic construct may become integrated in the host chromosomal DNA. The genetic construct may be linked to a vector.

The term “vector” refers to a polynucleotide molecule, usually double stranded DNA, which is used to transport the genetic construct into a host cell. The vector may be capable of replication in at least one additional host system, such as in a bacterial cell system, such as in E. coli or other suitable bacterial cell system.

The term “expression construct” refers to a genetic construct that includes the necessary elements that permit transcribing the insert polynucleotide molecule, and, optionally, translating the transcript into a polypeptide. An expression construct typically comprises in a 5′ to 3′ direction: a) a promoter functional in the host cell into which the construct will be transformed, b) the polynucleotide to be expressed, and c) a terminator functional in the host cell into which the construct will be transformed.

The term “coding region” or “open reading frame” (ORF) refers to the sense strand of a genomic DNA sequence or a cDNA sequence that is capable of producing a transcription product and/or a polypeptide under the control of appropriate regulatory sequences. The coding sequence may, in some cases, identified by the presence of a 5′ translation start codon and a 3′ translation stop codon. When inserted into a genetic construct, a “coding sequence” is capable of being expressed when it is operably linked to promoter and terminator sequences.

“Operably-linked” means that the sequenced to be expressed is placed under the control of regulatory elements that include promoters, tissue-specific regulatory elements, temporal regulatory elements, enhancers, repressors and terminators.

As used herein, “pg” means picogram, “ng” means nanogram, “ug” or “μg” mean microgram, “mg” means milligram, “ul” or “μl” mean microliter, “ml” means milliliter, “1” means liter.

Another feature of this invention is the expression of the DNA sequences disclosed herein. As is well known in the art, DNA sequences may be expressed by operatively linking them to an expression control sequence in an appropriate expression vector and employing that expression vector to transform an appropriate unicellular host. Such operative linking of a DNA sequence of this invention to an expression control sequence, of course, includes, if not already part of the DNA sequence, the provision of an initiation codon, ATG, in the correct reading frame upstream of the DNA sequence.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col El, pCR1, pBR322, pMB9 and their derivatives, plasmids such as RP4; phage DNAS, e.g., the numerous derivatives of phage k, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences—sequences that control the expression of a DNA sequence operatively linked to it—may be used in these vectors to express the DNA sequences of this invention. Such useful expression control sequences include, for example, promoters. Promoters suitable for expression in plants are well known and available. A tissue/organ preferred promoter is a promoter that drives expression of an operably linked polynucleotide in a particular tissue/organ at a higher level than in other tissues/organs. A tissue specific promoter is a promoter that drives expression of an operably linked polynucleotide specifically in a particular tissue/organ. Even with tissue/organ specific promoters, there is usually a small amount of expression in at least one other tissue. A tissue specific promoter is by definition also a tissue preferred promoter. Vegetative Tissue Specific Promoters—An example of a vegetative specific promoter is found in U.S. Pat. Nos. 6,229,067; and 7,629,454; and 7,153,953; and 6,228,643. Pollen Specific Promoters—An example of a pollen specific promoter is found in U.S. Pat. Nos. 7,141,424; and 5,545,546; and 5,412,085; and 5,086,169; and 7,667,097. Seed Specific Promoters—An example of a seed specific promoter is found in U.S. Pat. Nos. 6,342,657; and 7,081,565; and 7,405,345; and 7,642,346; and 7,371,928. Fruit Specific Promoters—An example of a fruit specific promoter is found in U.S. Pat. Nos. 5,536,653; and 6,127,179; and 5,608,150; and 4,943,674. Non-Photosynthetic Tissue Preferred Promoters—Non-photosynthetic tissue preferred promoters include those preferentially expressed in non-photosynthetic tissues/organs of the plant. Non-photosynthetic tissue preferred promoters may also include light repressed promoters. Light Repressed Promoters—An example of a light repressed promoter is found in U.S. Pat. Nos. 5,639,952 and in 5,656,496. Root Specific Promoters—An example of a root specific promoter is found in U.S. Pat. No. 5,837,848; and US 2004/0067506 and US 2001/0047525. Tuber Specific Promoters—An example of a tuber specific promoter is found in U.S. Pat. No. 6,184,443. Bulb Specific Promoters—An example of a bulb specific promoter is found in Smeets et al., (1997) Plant Physiol. 113:765-771. Rhizome Preferred Promoters—An example of a rhizome preferred promoter is found Seong Jang et al., (2006) Plant Physiol. 142:1148-1159. Endosperm Specific Promoters—An example of an endosperm specific promoter is found in U.S. Pat. No. 7,745,697. Photosynthetic Tissue Preferred Promoters—Photosynthetic tissue preferred promoters include those that are preferentially expressed in photosynthetic tissues of the plants. Photosynthetic tissues of the plant include leaves, stems, shoots and above ground parts of the plant. Photosynthetic tissue preferred promoters include light regulated promoters. Light Regulated Promoters—Numerous light regulated promoters are known to those skilled in the art and include for example chlorophyll a/b (Cab) binding protein promoters and Rubisco Small Subunit (SSU) promoters. An example of a light regulated promoter is found in U.S. Pat. No. 5,750,385. Light regulated in this context means light inducible or light induced.

A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, insect cells, and human cells and plant cells in tissue culture. Host cells may be derived from, for example, bacterial, fungal, yeast, insect, mammalian, algal or plant organisms. Host cells may also be synthetic cells. Preferred host cells are eukaryotic cells. A particularly preferred host cell is a plant cell, particularly a plant cell in a vegetative tissue of a plant.

A “transgenic plant” refers to a plant which contains new genetic material as a result of genetic manipulation or transformation. The new genetic material may be derived from a plant of the same species as the resulting transgenic plant or from a different species.

It will be understood that not all vectors, expression control sequences and hosts will function equally well to express the DNA sequences of this invention. Neither will all hosts function equally well with the same expression system. However, one skilled in the art will be able to select the proper vectors, expression control sequences, and hosts without undue experimentation to accomplish the desired expression without departing from the scope of this invention. For example, in selecting a vector, the host must be considered because the vector must function in it. The vector's copy number, the ability to control that copy number, and the expression of any other proteins encoded by the vector, such as antibiotic markers, will also be considered.

In selecting an expression control sequence, a variety of factors will normally be considered. These include, for example, the relative strength of the system, its controllability, and its compatibility with the particular DNA sequence or gene to be expressed, particularly as regards potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the DNA sequences to be expressed, and the ease of purification of the expression products.

Considering these and other factors a person skilled in the art will be able to construct a variety of vector/expression control sequence/host combinations that will express the DNA sequences of this invention on fermentation or in large scale animal culture.

The labels most commonly employed for studies with relevance to the present invention are known to one skilled in the art. Examples are radioactive elements, enzymes, chemicals which fluoresce when exposed to ultraviolet light, and others. A number of fluorescent materials are known and can be utilized as labels. These include, for example, fluorescein, rhodamine, auramine, Texas Red, AMCA blue and Lucifer Yellow. A particular detecting material is anti-rabbit antibody prepared in goats and conjugated with fluorescein through an isothiocyanate. The mutant stearoyl-acyl carrier protein (ACP) desaturase can also be labeled with a radioactive element or with an enzyme. The radioactive label can be detected by any of the currently available counting procedures. The preferred isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re. Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods.

The seeds of higher plants represent valuable factories capable of converting photosynthetically derived sugars into a variety of storage compounds, including oils. Oils are the most energy-dense plant reserves and fatty acids composing these oils represent an excellent nutritional source and supply humans with much of the calories and essential fatty acids required in their diet. These oils are increasingly being utilized as renewable alternatives to petroleum for the chemical industry and for biofuels. Plant oils represent a highly valuable agricultural commodity, the demand for which is increasing rapidly. Knowledge regarding seed oil production can be extensively exploited in the frame of breeding programs and approaches of metabolic engineering for oilseed crop improvement. Relevant aspects of this area of research for application and use include (1) the study of carbon metabolism responsible for the conversion of photosynthetically derived sugars into precursors for fatty acid biosynthesis, (2) the identification and characterization of the enzymatic actors allowing the production of the wide set of fatty acid structures found in seed oils, and (3) the investigation of the complex biosynthetic pathways leading to the production of storage lipids (waxes, triacylglycerols) (Baud S (2018) Plant Reproduction 31:213-235).

Stereoselective dihydroxylation reactions are important to the chemical industry (Borrell and Costas, 2017) since diols serve as valuable synthons. The osmium-based asymmetric dihydroxylation reaction (Crispino and Sharpless, 1993) is an example of controlled olefin oxidation and was (in part) recognized by the award of the 2001 Nobel Prize in Chemistry to its inventor, K. B. Sharpless. In addition, biocatalytic diol formation from aromatics by whole cell mutant Pseudomonas cultures has furnished a variety of enantiomerically pure cyclohexadiene-cis-diols (Hudlicky and Thorpe, 1996). There have also been efforts to develop iron-based biomimetic catalytic methodology for this reaction (Oloo and Que, 2015). Herein, is described an investigation of a “green chemical approach”: the castor Δ⁹18:0-ACP desaturase-mediated syn-dihydroxylation of an unactivated alkene.

Vicinal diol fatty acids (VDFAs) as used herein refers to fatty acids with two hydroxyl groups on adjacent carbons and may have uses as specialty fatty acids. Such functionalization may allow them to be used as activated feedstocks that can be chemically derivatized to form new compounds. VDFA have been identified in the oils of a number of plants including castor and Cardamine impatiens. While castor oil is abundant, the VDFA content is low in the approximate range of 1%. In contrast, VDFAs accumulate to approximately 25% in plants such as Cardamine impatiens, but the Cardamine impatiens itself has limited or low seed yield and there may be other properties that render it less than suitable for agronomic production of oil. It remains desirable to create a large-scale supply of VDFA in for example crop plants, microbes, or other living systems. This could be in production crop plants, microbes, or other living systems such as in microbes.

The present disclosure describes wild type stearoyl-ACP desaturase from castor bean, and its mutants for example, T117R and D280K capable of converting oleoyl-ACP, the normal product of stearoyl-ACP desaturase, to a vicinal diol, i.e., a saturated C18 fatty acid with hydroxy groups on adjacent C9 and C10 carbons, known as erythro 9, 10 dihydroxy stearate.

The methods encompassed in the invention include methods for synthesizing VDFA. A study was conducted with the object of obtaining stearoyl-ACP desaturase from Ricinus communis, i.e., castor bean, the primary function of which is to convert stearoyl-ACP, the 18C saturated fatty acid produced in plant plastids, to oleoyl-ACP, the corresponding C18 Δ9, monounsaturated fatty acid. In the present disclosure, a wild type stearoyl-ACP type desaturase, and mutants of this, notably, T117R and D280K are able to convert oleoyl-ACP, the normal product of the stearoyl-ACP desaturase, to a vicinal diol, i.e., a saturated C18 fatty acid with hydroxy groups on adjacent C9 and C10 carbons, known as erythro 9, 10 dihydroxy stearate. The diiron enzyme class of which the acyl-ACP desaturase is a member may perform monooxygenase chemistry while the present reaction described herein regarding the conversion of oleate to 9, 10 dihydroxystearate is a dioxygenase reaction. This means that the oxygens in both the 9, and 10 hydroxy groups originate from molecular oxygen.

Variation of the present reaction, i.e., the acyl-ACP mediated conversion of monounsaturated fatty acid to VDFA, may involve a variant of an acyl-Co-A integral membrane desaturase family, for example the ADS family of enzymes, such as ADS family enzyme from Arabidopsis thaliana. ADS genes are from a different polypeptide enzyme family lineage to the stearoyl ACP desaturases but have the same function. In embodiments of the invention, variants of either class of enzyme could result in the formation of VDFA. The Arabidopsis stearoyl ACP desaturase sequence may be distinct but related in enzymatic capability from that provided herein. They convert fatty acyl chains into monoenes, often C18 at the D9 position. Despite the distinct evolutionary origins of the soluble and membrane desaturases, they operate by employing a diiron center to activate a molecular oxygen by similar chemical mechanisms. Thus, it is possible that the enzymes, or variants thereof could perform similar chemistries, one of which could be to form VDFA from cis monoenes. In embodiments of all aspects of the invention, the mutant plant enzyme polypeptide may be an acyl-Co-A integral membrane desaturase enzyme polypeptide. In embodiments of all aspects of the invention, the mutant plant enzyme polypeptide may be an acyl-Co-A integral membrane desaturase enzyme polypeptide, and may be an Arabidopsis stearoyl ACP desaturase sequence.

Stearoyl-ACP desaturase (SAD) is a plastid-localized soluble desaturase that catalyzes the conversion of stearic acid (18:0) to oleic acid, which plays a key role in determining the ratio of saturated to unsaturated fatty acids. In enzymology, an acyl-[acyl-carrier-protein] desaturase (EC 1.14.19.2) is an enzyme that catalyzes the chemical reaction stearoyl-[acyl-carrier-protein]+reduced acceptor+O₂↔oleoyl-[acyl-carrier-protein]+acceptor+2 H₂O

The systematic name of this enzyme class is acyl-[acyl-carrier-protein], hydrogen-donor:oxygen oxidoreductase. Other names in common use include stearyl acyl carrier protein desaturase, and stearyl-ACP desaturase. The enzyme participates in polyunsaturated fatty acid biosynthesis and employs one cofactor, ferredoxin. Ferredoxins (from Latin ferrum: iron+redox, often abbreviated “fd”) are iron-sulfur proteins that mediate electron transfer in a range of metabolic reactions. This enzyme class plays a critical role in the biosynthesis of unsaturated fatty acids in plants, and the enzymes are very specific to their substrates (Behrouzian B, Buist B H (2002) Curr Opinion in Chemical Biology 6(5):577-582). A common theme in recent research has been to identify uncommon desaturases in various plants and isolate their genetic code (Shanklin J and Cahoon E (1998) Ann Rev Plant Phys and Plant Molecular Biol 49:611-641; Schultz D et al (2000) Plant Physiology 124(2):681-692). In particular, sequences encoding these desaturases can then be inserted into model cells (such as Escherichia coli) and up-regulated through metabolic engineering to skew the composition of oils produced by the model cells (Cahoon E, Mills L and Shanklin J (1996) J Bact 178(3):936-939). This would become particularly important and applicable if possible to successfully synthesize so-called Omega-3 fatty acids or other nutraceutical products from basic saturated fatty acids, and extract or isolate them from their hosts.

The full length R. cumminus (castor) stearoyl-ACP desaturase amino acid sequence is provided below (SEQ ID NO:1) Genbank M59857.1, protein ID AAA74692.1). The methionine start is shown in bold. Amino acids corresponding to the exemplary amino acid variant and mutant locations are underlined, particularly T117, G188 and D280.

| | |
|---|---|
| 1 | FRQITKNQKK KVRKKTMALK LNPFLSQTQK LPSFALPPMA STRSPKFYMA STLKSGSKEV |
| 61 | ENLKKPFMPP REVHVQVTHS MPPQKIEIFK SLDNWAEENI LVHLKPVEKC WQPQDFLPDP |
| 121 | ASDGFDEQVR ELRERAKEIP DDYFVVLVGD MITEEALPTY QTMLNTLDGV RDETGASPTS |
| 181 | WAIWTRAWTA EENRHGDLLN KYLYLSGRVD MRQIEKTIQY LIGSGMDPRT ENSPYLGFIY |
| 241 | TSFQERATFI SHGNTARQAK EHGDIKLAQI CGTIAADEKR HETAYTKIVE KLFEIDPDGT |
| 301 | VLAFADMMRK KISMPAHLMY DGRDDNLFDH FSAVAQRLGV YTAKDYADIL EFLVGRWKVD |
| 361 | KLTGLSAEGQ KAQDYVCRLP PRIRRLEERA QGRAKEAPTM PFSWIFDRQV KL |

The R. cumminus (castor) stearoyl-ACP desaturase sequence starting with the bolded methionine corresponds to SEQ ID NO:7.

| | |
|---|---|
| 1 | MALKLNPFLS QTQKLPSFAL PPMASTRSPK FYMASTLKSG SKEVENLKKP FMPPREVHVQ |
| 61 | VTHSMPPQKI EIFKSLDNWA EENILVHLKP VEKCWQPQDF LPDPASDGFD EQVRELRERA |
| 121 | KEIPDDYFVV LVGDMITEEA LPTYQTMLNT LDGVRDETGA SPTSWAIWTR AWTAEENRHG |
| 181 | DLLNKYLYLS GRVDMRQIEK TIQYLIGSGM DPRTENSPYL GFIYTSFQER ATFISHGNTA |
| 241 | RQAKEHGDIK LAQICGTIAA DEKRHETAYT KIVEKLFEID PDGTVLAFAD MMRKKISMPA |
| 301 | HLMYDGRDDN LFDHFSAVAQ RLGVYTAKDY ADILEFLVGR WKVDKLTGLS AEGQKAQDYV |
| 361 | CRLPPRIRRL EERAQGRAKE APTMPFSWIF DRQVKL |

Exemplary nucleic acid sequence encoding the R. cumminus (castor) stearoyl-ACP desaturase sequence of SEQ ID NO:1 is provided below (SEQ ID NO:14).

| | |
|---|---|
| 1 | ttccggcaaa taacaaaaaa ccaaaagaaa aaggtaagaa aaaaaacaat ggctctcaag |
| 61 | ctcaatcctt tcctttctca aacccaaaag ttaccttctt tcgctcttcc accaatggcc |
| 121 | agtaccagat ctcctaagtt ctacatggcc tctaccctca agtctggttc taaggaagtt |
| 181 | gagaatctca agaagccttt catgcctcct cgggaggtac atgttcaggt tacccattct |
| 241 | atgccacccc aaaagattga gatctttaaa tccctagaca attgggctga ggagaacatt |
| 301 | ctggttcatc tgaagccagt tgagaaatgt tggcaaccgc aggatttttt gccagatccc |
| 361 | gcctctgatg gatttgatga gcaagtcagg gaactcaggg agagagcaaa ggagattcct |
| 421 | gatgattatt ttgttgtttt ggttggagac atgataacgg aagaagccct tcccacttat |
| 481 | caaacaatgc tgaatacctt ggatggagtt cgggatgaaa caggtgcaag tcctacttct |
| 541 | tgggcaattt ggacaagggc atggactgcg gaagagaata gacatggtga cctcctcaat |
| 601 | aagtatctct acctatctgg acgagtggac atgaggcaaa ttgagaagac aattcaatat |
| 661 | ttgattggtt caggaatgga tccacggaca gaaaacagtc cataccttgg gttcatctat |
| 721 | acatcattcc aggaaagggc aaccttcatt tctcatggga acactgcccg acaagccaaa |
| 781 | gagcatggag acataaagtt ggctcaaata tgtggtacaa ttgctgcaga tgagaagcgc |
| 841 | catgagacag cctacacaaa gatagtggaa aaactctttg agattgatcc tgatggaact |
| 901 | gttttggctt ttgctgatat gatgagaaag aaaatttcta tgcctgcaca cttgatgtat |
| 961 | gatggccgag atgataatct ttttgaccac ttttcagctg ttgcgcagcg tcttggagtc |
| 1021 | tacacagcaa aggattatgc agatatattg gagttcttgg tgggcagatg gaaggtggat |
| 1081 | aaactaacgg gcctttcagc tgagggacaa aaggctcagg actatgtttg tcggttacct |
| 1141 | ccaagaatta gaaggctgga agagagagct caaggaaggg caaaggaagc acccaccatg |
| 1201 | cctttcagct ggattttcga taggcaagtg aagctgtag |

The castor stearoyl-ACP desaturase sequence is post-translationally modified. A 33 amino-acid N-terminal transit peptide sequence that directs the desaturase to the plastid and is removed after transport into the plastid. The plastid localized polypeptide is shorter in length from the full length sequence as originally reported in Shanklin and Sommerville (1991) PNAS USA 88:2510-2514.

The enzyme sequence as active and present in the plastid corresponds to the following sequence (SEQ ID NO:2) (amino acids corresponding to the mutant locations T117, G188 and D280 are underlined)

| | |
|---|---|
| 1 | ASTLKSGSKE VENLKKPFMP PREVHVQVTH SMPPQKIEIF KSLDNWAEEN ILVHLKPVEK |
| 61 | CWQPQDFLPD PASDGFDEQV RELRERAKEI PDDYFVVLVG DMITEEALPT YQTMLNTLDG |
| 121 | VRDETGASPT SWAIWTRAWT AEENRHGDLL NKYLYLSGRV DMRQIEKTIQ YLIGSGMDPR |
| 181 | TENSPYLGFI YTSFQERATF ISHGNTARQA KEHGDIKLAQ ICGTIAADEK RHETAYTKIV |
| 241 | EKLFEIDPDG TVLAFADMMR KKISMPAHLM YDGRDDNLFD HFSAVAQRLG VYTAKDYADI |
| 301 | LEFLVGRWKV DKLTGLSAEG QKAQDYVCRL PPRIRRLEER AQGRAKEAPT MPFSWIFDRQ |
| 361 | VKL |

The castor (R. communis) individual variants are T117R (SEQ ID NO:3), G188L (SEQ ID NO:4) and D280K (SEQ ID NO:5). In addition, a double mutant T117R/D280K (SEQ ID NO:6) is also contemplated and provided as an embodiment of the invention. These sequences are provided below:

| T117R | |
|---|---|
| (SEQ ID NO: 3) | |
| 1 | ASTLKSGSKE VENLKKPFMP PREVHVQVTH SMPPQKIEIF KSLDNWAEEN ILVHLKPVEK |
| 61 | CWQPQDFLPD PASDGFDEQV RELRERAKEI PDDYFVVLVG DMITEEALPT YQTMLNRLDG |
| 121 | VRDETGASPT SWAIWTRAWT AEENRHGDLL NKYLYLSGRV DMRQIEKTIQ YLIGSGMDPR |
| 181 | TENSPYLGFI YTSFQERATF ISHGNTARQA KEHGDIKLAQ ICGTIAADEK RHETAYTKIV |
| 241 | EKLFEIDPDG TVLAFADMMR KKISMPAHLM YDGRDDNLFD HFSAVAQRLG VYTAKDYADI |
| 301 | LEFLVGRWKV DKLTGLSAEG QKAQDYVCRL PPRIRRLEER AQGRAKEAPT MPFSWIFDRQ |
| 361 | VKL |
| G188L | |
| (SEQ ID NO: 4) | |
| 1 | ASTLKSGSKE VENLKKPFMP PREVHVQVTH SMPPQKIEIF KSLDNWAEEN ILVHLKPVEK |
| 61 | CWQPQDFLPD PASDGFDEQV RELRERAKEI PDDYFVVLVG DMITEEALPT YQTMLNTLDG |
| 121 | VRDETGASPT SWAIWTRAWT AEENRHGDLL NKYLYLSGRV DMRQIEKTIQ YLIGSGMDPR |
| 181 | TENSPYLLFI YTSFQERATF ISHGNTARQA KEHGDIKLAQ ICGTIAADEK RHETAYTKIV |
| 241 | EKLFEIDPDG TVLAFADMMR KKISMPAHLM YDGRDDNLFD HFSAVAQRLG VYTAKDYADI |
| 301 | LEFLVGRWKV DKLTGLSAEG QKAQDYVCRL PPRIRRLEER AQGRAKEAPT MPFSWIFDRQ |
| 361 | VKL |
| D280K | |
| (SEQ ID NO: 5) | |
| 1 | ASTLKSGSKE VENLKKPFMP PREVHVQVTH SMPPQKIEIF KSLDNWAEEN ILVHLKPVEK |
| 61 | CWQPQDFLPD PASDGFDEQV RELRERAKEI PDDYFVVLVG DMITEEALPT YQTMLNTLDG |
| 121 | VRDETGASPT SWAIWTRAWT AEENRHGDLL NKYLYLSGRV DMRQIEKTIQ YLIGSGMDPR |
| 181 | TENSPYLGFI YTSFQERATF ISHGNTARQA KEHGDIKLAQ ICGTIAADEK RHETAYTKIV |
| 241 | EKLFEIDPDG TVLAFADMMR KKISMPAHLM YDGRDDNLFK HFSAVAQRLG VYTAKDYADI |
| 301 | LEFLVGRWKV DKLTGLSAEG QKAQDYVCRL PPRIRRLEER AQGRAKEAPT MPFSWIFDRQ |
| 361 | VKL |
| T117R/D280K | |
| (SEQ ID NO: 6) | |
| 1 | ASTLKSGSKE VENLKKPFMP PREVHVQVTH SMPPQKIEIF KSLDNWAEEN ILVHLKPVEK |
| 61 | CWQPQDFLPD PASDGFDEQV RELRERAKEI PDDYFVVLVG DMITEEALPT YQTMLNRLDG |
| 121 | VRDETGASPT SWAIWTRAWT AEENRHGDLL NKYLYLSGRV DMRQIEKTIQ YLIGSGMDPR |
| 181 | TENSPYLGFI YTSFQERATF ISHGNTARQA KEHGDIKLAQ ICGTIAADEK RHETAYTKIV |
| 241 | EKLFEIDPDG TVLAFADMMR KKISMPAHLM YDGRDDNLFK HFSAVAQRLG VYTAKDYADI |
| 301 | LEFLVGRWKV DKLTGLSAEG QKAQDYVCRL PPRIRRLEER AQGRAKEAPT MPFSWIFDRQ |
| 361 | VKL |

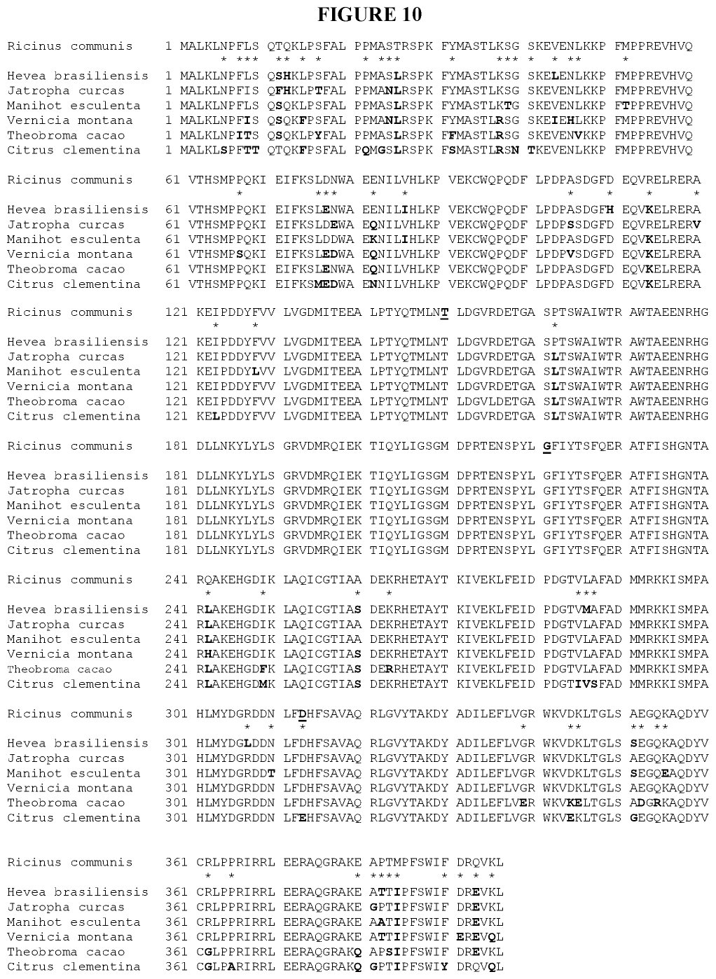

Examples of alternative and varied plant stearoyl-ACP desaturase sequences suitable to be mutated as described herein and for use in the invention are well known and available to one skilled in the art including in public sequence databases. For example, a BLAST search of the NCBI sequence protein database with the castor stearoyl-ACP desaturase sequence provided herein (SEQ ID NO:1 or SEQ ID NO:7 or the plastid sequence SEQ ID NO:2) will result in numerous similar plant stearoyl-ACP desaturase sequences being generated as search output, with numerous alternative plant sequences having sequence identity with the castor stearoyl-ACP desaturase sequence ranging from 96% to about 88%. Various related or distinct plant species stearoyl-ACP desaturase proteins are therefore known and available. Exemplary sequences from other plants can readily be identified and compared or aligned with the castor plant stearoyl-ACP desaturase (SEQ ID NO:1, SEQ ID NO:7, SEQ ID NO:2) hereof so as to provide comparable corresponding amino acids to generate further or alternative stearoyl-ACP desaturase enzyme sequences for mutation and variation for use and application in generating vicinal diol(s) in accordance with the invention. One skilled in the art can readily identify and compare other plant stearoyl-ACP desaturase enzyme sequences which may be suitable for mutation or variation in line with the present invention. For example, a BLAST sequence with the castor amino acid sequence of either SEQ ID NO:1, 2 or 7 will identify homologous enzyme sequences from other plant species and genus. An example of an alignment of the R. communis SEQ ID NO:7 with comparable plant sequences is provided herein for example in FIG. 10. Amino acids that vary from the castor sequence in other plant species sequences are indicated with an asterisk. Notably, the T117 amino acid sequence is not altered in any of the alternative plant sequences shown. Neither is the G188 sequence. The D280 amino acid is conservatively varied to an E in a citrus plant sequence.

The exemplary mutation of threonine (T) 117 to an arginine (R) amino acid is a non-conservative change. Similarly, the exemplary mutation of glycine (G) 188 to a leucine (L) and of aspartic acid (D) 280 to lysine (K) are non-conservative changes. Non conservative amino acid substitutions of plastid stearoyl-ACP desaturase enzyme polypeptide at one or more amino acid corresponding to the plastid enzyme amino acid 117 and/or 280 are contemplated herein to provide mutant stearoyl-ACP desaturase enzyme polypeptide(s) of the invention and of use in the invention.

Other comparable stearoyl-ACP desaturase sequences, demonstrating on the order of 90-96% amino acid identity to the sequence of the castor stearoyl-ACP desaturase include but are not limited to, for example: Herrania umbratica, stearoyl-(acyl-carrier-protein) 9-desaturase, chloroplastic (XP_021290741.1); Triadica sebifera, stearoyl-ACP desaturase (ABNI3874.1); Citrus unshui, hypothetical protein CUMW_084590 (GAY44796.1); Idesia polycarpa, stearoyl-(acyl-carrier-protein) 9-desaturase (QDX46951); Pistacia vera, stearoyl-(acyl-carrier-protein) 9-desaturase (XP_031260711.1); Venicia forii (tung tree for tung oil) SAD (ADC32803.1); Sesamum indicum (sesame plant), stearoyl-acyl carrier protein desaturase (BAA07681.1).

In accordance with the present invention, variant or mutant enzyme polypeptides of enzyme polypeptides having at least 85% amino acid identity or at least 90% amino acid identity with the castor stearoyl-ACP type deasaturase polypeptide are provided wherein one or more amino acid substitution is introduced and wherein the variant or mutant desaturase is capable of converting oleoyl-ACP to a vicinal diol. In accordance with the present invention, variant or mutant stearoyl-ACP type deasaturase polypeptides are provided wherein one or more amino acid substitution is introduced and wherein the variant or mutant desaturase is capable of converting oleoyl-ACP to a vicinal diol. In accordance with the invention, the variant or mutant enzyme polypeptides or the variant or mutant stearoyl-ACP type deasaturase is capable of converting oleoyl-ACP to a vicinal diol, such that vicinal diol accumulates at an increased level, increasing by 10 fold or greater, such that at least 10%, up to 15%, up to 20%, up to 25%, up to 30% in the plant seed oil, for example in castor oil.

Plant seed oils are commercially significant and relevant. Seed oil is a vegetable oil that is obtained from the seed (endosperm) of some plants, rather than the fruit (pericarp). Most vegetable oils are seed oils. Some common examples are sunflower oil, canola oil, and sesame oil. Seed oil plants of use and commercial application include: almond, argan, borage, canola, castor, cherry, coconut, corn, cotton, flax, grape, hemp, jojoa, macadamia, mango, mustard, neem, oil palm, rapeseed, safflower, sesame, shea, sunflower, tonka bean, tung.

In an embodiment, the mutant or variant stearoyl-ACP desaturase is capable of accumulating a novel product erythro-9,10-dihydroxystearate.

The invention provides a mutant plant stearoyl-acyl carrier protein (ACP) desaturase polypeptide capable of catalyzing the conversion of oleoyl-ACP to erythro 9,10 dihydroxy stearate, wherein one or more amino acid is substituted and wherein the conversion of oleoyl-ACP to erythro 9,10 dihydroxy stearate is increased. In an embodiment, the conversion of oleoyl-ACP to erythro 9,10 dihydroxy stearate is increased by at least 10 fold compared the wild type or native, non mutant plant stearoyl-acyl carrier protein (ACP) desaturase polypeptide. In an embodiment, the mutant stearoyl-acyl carrier protein (ACP) desaturase polypeptide generates 9,10 dihydroxy stearate as a component of castor oil at or up to at least 10% of the total fatty acids. In an embodiment, the mutant stearoyl-acyl carrier protein (ACP) desaturase polypeptide generates 9,10 dihydroxy stearate as a component of castor oil at or up to at least 15% of the total fatty acids. In an embodiment, the mutant stearoyl-acyl carrier protein (ACP) desaturase polypeptide generates 9,10 dihydroxy stearate as a component of castor oil at or up to at least 20% of the total fatty acids. In an embodiment, the mutant stearoyl-acyl carrier protein (ACP) desaturase polypeptide generates 9,10 dihydroxy stearate as a component of castor oil at or up to at least 25% of the total fatty acids.

The invention provides a mutant plant diiron enzyme polypeptide capable of a dioxygenase reaction mechanism wherein a double bond is converted to a vicinal diol.

In an embodiment, the mutant plant diiron enzyme polypeptide is a mutant stearoyl-acyl carrier protein (ACP) desaturase polypeptide capable of catalyzing the conversion of oleoyl-ACP to erythro 9,10 dihydroxy stearate comprising:

(a) an amino acid replacement of the threonine (T) at amino acid residue 117 of the processed plastid polypeptide sequence and corresponding to residue 117 of SEQ ID NO: 2 or of the amino acid at the corresponding position in a plant stearoyl-ACP desaturase polypeptide;

(b) an amino acid replacement of the aspartic acid (D) at amino acid residue 280 of the processed plastid polypeptide sequence and corresponding to residue 280 of SEQ ID NO: 2 or of the amino acid at the corresponding position in a plant stearoyl-ACP desaturase polypeptide; or

(c) an amino acid replacement of the threonine (T) at amino acid residue 117 of the processed plastid polypeptide sequence and corresponding to residue 117 of SEQ ID NO: 2 or of the amino acid at the corresponding position in a plant stearoyl-ACP desaturase polypeptide and an amino acid replacement of the aspartic acid (D) at amino acid residue 280 of the processed plastid polypeptide sequence and corresponding to residue 280 of SEQ ID NO: 2 or of the amino acid at the corresponding position in a plant stearoyl-ACP desaturase polypeptide.

In an embodiment, the mutant plant diiron enzyme polypeptide is a mutant plant enzyme polypeptide having at least 85% amino acid identity or at least 90% amino acid identity to the castor stearoyl-acyl carrier protein (ACP) desaturase polypeptide (such as that of SEQ ID NO:2) and is capable of catalyzing the conversion of substrate such as oleoyl-ACP or other applicable substrate to a vicinal diol for example or such as erythro 9,10 dihydroxy stearate comprising:

(a) an amino acid replacement of the threonine (T) at amino acid residue 117 of the processed plastid polypeptide sequence and corresponding to residue 117 of SEQ ID NO: 2 or of the amino acid at the corresponding position in a plant stearoyl-ACP desaturase polypeptide;

(b) an amino acid replacement of the aspartic acid (D) at amino acid residue 280 of the processed plastid polypeptide sequence and corresponding to residue 280 of SEQ ID NO: 2 or of the amino acid at the corresponding position in a plant stearoyl-ACP desaturase polypeptide; or

(c) an amino acid replacement of the threonine (T) at amino acid residue 117 of the processed plastid polypeptide sequence and corresponding to residue 117 of SEQ ID NO: 2 or of the amino acid at the corresponding position in a plant stearoyl-ACP desaturase polypeptide and an amino acid replacement of the aspartic acid (D) at amino acid residue 280 of the processed plastid polypeptide sequence and corresponding to residue 280 of SEQ ID NO: 2 or of the amino acid at the corresponding position in a plant stearoyl-ACP desaturase polypeptide.

In an embodiment, the mutant plant enzyme polypeptide or the mutant stearoyl-acyl carrier protein (ACP) desaturase polypeptide comprises an amino acid replacement at residue 117 or its corresponding position wherein the amino acid threonine or such other hydroxylic amino acid or amino acid having an uncharged polar R group is replaced with a basic amino acid or charged or nonpolar R group.

In an embodiment, the mutant plant enzyme polypeptide or mutant stearoyl-acyl carrier protein (ACP) desaturase polypeptide comprises an amino acid replacement at residue 280 or its corresponding position wherein the amino acid aspartic acid or such other acidic amino acid or amino acid having a polar R group is replaced with a basic amino acid or uncharged or nonpolar R group.

In an embodiment, the mutant plant enzyme polypeptide or mutant stearoyl-acyl carrier protein (ACP) desaturase polypeptide comprises an amino acid replacement at residue 117 or its corresponding position wherein the amino acid threonine or such other hydroxylic amino acid or amino acid having an uncharged polar R group is replaced with a basic amino acid or charged or nonpolar R group and further comprises an amino acid replacement at residue 280 or its corresponding position wherein the amino acid aspartic acid or such other acidic amino acid or amino acid having a polar R group is replaced with a basic amino acid or uncharged or nonpolar R group.

In an embodiment, the mutant plant enzyme polypeptide or mutant stearoyl-acyl carrier protein (ACP) desaturase polypeptide comprises an amino acid replacement at residue 117 or its corresponding position wherein the amino acid threonine is replaced with a basic amino selected from arginine, lysine and histidine. In an embodiment, the mutant polypeptide comprises an amino acid replacement at residue 117 or its corresponding position wherein the amino acid threonine is replaced with an arginine.

In an embodiment, the mutant plant enzyme polypeptide or mutant stearoyl-acyl carrier protein (ACP) desaturase polypeptide comprises an amino acid replacement at residue 280 or its corresponding position wherein the amino acid aspartic acid is replaced with a basic amino selected from arginine, lysine and histidine. In an embodiment, the mutant polypeptide comprises an amino acid replacement at residue 280 or its corresponding position wherein the amino acid threonine is replaced with a lysine.

In an embodiment, the mutant stearoyl-acyl carrier protein (ACP) desaturase polypeptide catalyzes the conversion of oleoyl-ACP to erythro 9,10 dihydroxy stearate and generates at least 10 fold more erythro 9,10 dihydroxy stearate than the wild type or native, non mutant plant stearoyl-acyl carrier protein (ACP) desaturase polypeptide.

In an embodiment, the mutant plant enzyme polypeptide catalyzes the conversion of substrate to a vicinal diol and generates at least 10 fold more vicinal diol than the wild type or native, non mutant plant enzyme polypeptide. In an embodiment, the mutant plant enzyme polypeptide catalyzes the conversion of substrate to a vicinal diol and generates at least 20 fold more vicinal diol than the wild type or native, non mutant plant enzyme polypeptide. The mutant plant enzyme polypeptide catalyzes the conversion of substrate to a vicinal diol and generates at least 30 fold more vicinal diol than the wild type or native, non mutant plant enzyme polypeptide. The mutant plant enzyme polypeptide catalyzes the conversion of substrate to a vicinal diol and generates at least 40 fold more vicinal diol than the wild type or native, non mutant plant enzyme polypeptide. the mutant plant enzyme polypeptide catalyzes the conversion of substrate to a vicinal diol and generates at least 50 fold more vicinal diol than the wild type or native, non mutant plant enzyme polypeptide. The mutant plant enzyme polypeptide catalyzes the conversion of substrate to a vicinal diol and generates at least 10 fold, 20 fold, 30 fold, 40 fold, 50 fold, 60 fold, 80 fold, 100 fold more vicinal diol than the wild type or native, non mutant plant enzyme polypeptide. In an embodiment, the mutant stearoyl-acyl carrier protein (ACP) desaturase polypeptide catalyzes the conversion of oleoyl-ACP to erythro 9,10 dihydroxy stearate and generates at least 10 fold more erythro 9,10 dihydroxy stearate than the wild type or native, non mutant plant stearoyl-acyl carrier protein (ACP) desaturase polypeptide. In an embodiment, the mutant stearoyl-acyl carrier protein (ACP) desaturase polypeptide catalyzes the conversion of oleoyl-ACP to y 9,10 dihydroxy stearate and generates at least 10 fold more erythro 9,10 dihydroxy stearate than the wild type or native, non mutant plant stearoyl-acyl carrier protein (ACP) desaturase polypeptide. In an embodiment, the mutant stearoyl-acyl carrier protein (ACP) desaturase polypeptide catalyzes the conversion of oleoyl-ACP to y 9,10 dihydroxy stearate and generates at least 20 fold more erythro 9,10 dihydroxy stearate than the wild type or native, non mutant plant stearoyl-acyl carrier protein (ACP) desaturase polypeptide.

In an embodiment, the mutant stearoyl-acyl carrier protein (ACP) desaturase polypeptide catalyzes the conversion of oleoyl-ACP to erythro 9,10 dihydroxy stearate and generates at least 30 fold more erythro 9,10 dihydroxy stearate than the wild type or native, non mutant plant stearoyl-acyl carrier protein (ACP) desaturase polypeptide. In an embodiment, the mutant stearoyl-acyl carrier protein (ACP) desaturase polypeptide catalyzes the conversion of oleoyl-ACP to erythro 9,10 dihydroxy stearate and generates at least 40 fold more erythro 9,10 dihydroxy stearate than the wild type or native, non mutant plant stearoyl-acyl carrier protein (ACP) desaturase polypeptide. In an embodiment, the mutant stearoyl-acyl carrier protein (ACP) desaturase polypeptide catalyzes the conversion of oleoyl-ACP to erythro 9,10 dihydroxy stearate and generates at least 50 fold more erythro 9,10 dihydroxy stearate than the wild type or native, non mutant plant stearoyl-acyl carrier protein (ACP) desaturase polypeptide. In an embodiment, the mutant stearoyl-acyl carrier protein (ACP) desaturase polypeptide catalyzes the conversion of oleoyl-ACP to erythro 9,10 dihydroxy stearate and generates at least 10 fold, 20 fold, 30 fold, 40 fold, 50 fold, 60 fold, 80 fold, 100 fold more erythro 9,10 dihydroxy stearate than the wild type or native, non mutant plant stearoyl-acyl carrier protein (ACP) desaturase polypeptide.

In an embodiment, the vicinal diol represents at least 10%, at least 20%, at least 30%, at least 40%, at least 50% of the product generated by the mutant enzyme polypeptide or the mutant stearoyl-acyl carrier protein (ACP) desaturase polypeptide. In an embodiment, the erythro 9,10 dihydroxy stearate represents at least 10%, at least 20%, at least 30%, at least 40%, at least 50% of the product generated by the mutant enzyme polypeptide or the mutant stearoyl-acyl carrier protein (ACP) desaturase polypeptide.

Nucleic acids or polynucleotides encoding the mutant plant enzyme polypeptide or mutant stearoyl-acyl carrier protein (ACP) desaturase polypeptides are also provided. The invention provides an isolated nucleic acid encoding the polypeptide, in particular the mutant plant enzyme polypeptide or mutant stearoyl-acyl carrier protein (ACP) desaturase polypeptide. In an embodiment, nucleic acid is provided encoding one or more mutant plant enzyme polypeptide or mutant stearoyl-acyl carrier protein (ACP) desaturase as described herein, including mutant plant enzyme polypeptide or mutant stearoyl-acyl carrier protein (ACP) desaturase having one or more amino acid replacement or substitution as provided herein. In an embodiment, nucleic acid is provided encoding one or more mutant plant enzyme polypeptide as described herein, including mutant plant enzyme polypeptide having a replacement at plastid enzyme amino acid 117 or the corresponding position thereof as provided herein. In an embodiment, nucleic acid is provided encoding one or more mutant plant enzyme polypeptide as described herein, including mutant plant enzyme polypeptide having a replacement at plastid enzyme amino acid 280 or the corresponding position thereof as provided herein. In an embodiment, nucleic acid is provided encoding one or more mutant stearoyl-acyl carrier protein (ACP) desaturase as described herein, including mutant stearoyl-acyl carrier protein (ACP) desaturase having a replacement at plastid enzyme amino acid 117 or the corresponding position thereof as provided herein. In an embodiment, nucleic acid is provided encoding one or more mutant stearoyl-acyl carrier protein (ACP) desaturase as described herein, including mutant stearoyl-acyl carrier protein (ACP) desaturase having a replacement at plastid enzyme amino acid 280 or the corresponding position thereof as provided herein.

In a further embodiment the polynucleotide encodes a fusion protein including the modified or mutant plant enzyme polypeptide or the modified or mutant stearoyl-acyl carrier protein (ACP) desaturase fused to a protein of interest.

The invention provides an expression construct comprising a polynucleotide of the invention. In one embodiment the polynucleotide in the construct is operably linked to a promoter sequence. In one embodiment the promoter sequence is capable of driving expression of the polynucleotide in a vegetative tissue of a plant. In another embodiment the promoter sequence is capable of driving expression of the polynucleotide in a seed of a plant. In a further embodiment the promoter sequence is capable of driving expression of the polynucleotide in the pollen of a plant. In a further embodiment the promoter sequence is capable of driving expression of the polynucleotide in a bacterial cell or yeast cell.

The invention includes a recombinant vector comprising the nucleic acid of the invention. In another aspect, the invention provides a construct containing a polynucleotide that encodes a mutant plant enzyme polypeptide or mutant stearoyl-acyl carrier protein (ACP) desaturase as provided herein. In various embodiments, the construct can be linked to a promoter sequence capable of driving its expression in various host cells. As such, the invention also provides use of the constructs to induce a host cell to express a modified or mutant plant enzyme polypeptide or a modified or mutant stearoyl-acyl carrier protein (ACP) desaturase. In yet another embodiment the construct is located in an appropriate position and orientation of a suitable functional endogenous promoter such that the expression of the construct occurs. In various embodiments, the construct can be expressed in a bacterial, plant, fungal or algal cell. In one embodiment where the construct is expressed in a plant cell, the cell may be of vegetative, seed, pollen or fruit tissue.

In another aspect the invention provides a host cell comprising a construct and mutant plant enzyme polypeptide or mutant stearoyl-acyl carrier protein (ACP) desaturase of the invention. In an aspect the invention provides a host cell genetically modified to comprise a polynucleotide of the invention. In a further aspect the invention provides a host cell genetically modified to express a polynucleotide of the invention. In a further embodiment, host cell(s) comprising the vector are provided. A host cell and host cells recombinantly engineered to heterologously produce the mutant plant enzyme polypeptide or mutant stearoyl-acyl carrier protein (ACP) desaturase polypeptide are provided herein. In embodiments, host cell(s) are recombinantly engineered to produce mutant plant enzyme polypeptide or mutant stearoyl-acyl carrier protein (ACP) desaturase by introducing nucleic acid encoding the mutant polypeptide.

The host cell may be a plant cell, bacterial cell or yeast cell or fungi. The host cell may be a plant cell. The host cell may be a bacterial cell. The instant examples describe expression of mutant stearoyl-acyl carrier protein (ACP) desaturase in bacteria, particularly in E. coli and analysis and assessment of the expressed protein, including its activity and enzymatic products upon incubation in vitro with substrate. One skilled in the art has available and known methods and systems for expressing plant or other enzymes in bacterial systems etc. for the purpose of generating certain fatty acids or enzymatic products. Previous studies have been reported of modifying the fatty acid composition of bacteria (such as Escherichia coli) by coexpression of a plant acyl-acyl carrier protein desaturase and ferredoxin (e.g. Cahoon E B et al (1996) J Bacteriology 178(3):936-939)

The nucleic acid may be operably linked to a promoter sequence. Suitable promoters for assessment or production in any applicable host or cell system are known and available to one skilled in the art. The promoter sequence may be capable of driving expression of the nucleic acid sequence in a bacterial cell. The promoter sequence may be capable of driving expression of the nucleic acid sequence in a vegetative tissue of a plant. In one aspect the promoter sequence is capable of driving expression of the nucleic acid sequence in a seed of a plant or in the pollen of a plant. The promoter sequence may be capable of driving expression of the polynucleotide in a bacterial cell or in a yeast cell.

The nucleic acid or polynucleotide of the invention may be operably linked to a promoter sequence. In an aspect, the promoter is suitable and applicable for expression in plants. In an aspect, the promoter is a constitutive promoter. In an aspect, the promoter is an inducible promoter. In an aspect, the promoter is a plant specific promoter, or a promoter directing expression in leaves, tissues or seeds of a plant. In an aspect, the promoter sequence is capable of driving expression of the nucleic acid sequence in a vegetative tissue of a plant. In one embodiment the promoter sequence is capable of driving expression of the nucleic acid sequence in a seed of a plant. In one embodiment the promoter sequence is capable of driving expression of the nucleic acid sequence in the pollen of a plant. In aspects, the promoter may be the constitutive promoter 35S or may be a seed promoter, particularly a strong seed promoter such as the promoter for the gene phaseolin.

The promoters suitable for use in the constructs of this invention are functional in a cell, tissue or organ of a monocot or dicot plant and include cell-, tissue- and organ-specific promoters, cell cycle specific promoters, temporal promoters, inducible promoters, constitutive promoters that are active in most plant tissues, and recombinant promoters. Choice of promoter will depend upon the temporal and spatial expression of the cloned polynucleotide, so desired. The promoters may be those normally associated with a transgene of interest, or promoters which are derived from genes of other plants, viruses, and plant pathogenic bacteria and fungi. Those skilled in the art will, without undue experimentation, be able to select promoters that are suitable for use in modifying and modulating plant traits using genetic constructs comprising the polynucleotide sequences of the invention. Examples of constitutive plant promoters include the CaMV 35S promoter, the nopaline synthase promoter and the octopine synthase promoter, and the Ubi 1 promoter from maize. Plant promoters which are active in specific tissues, respond to internal developmental signals or external abiotic or biotic stresses are described in the scientific literature. Exemplary promoters are described, e.g., in WO 02/00894, which is herein incorporated by reference.

A host plant comprising a vector encoding the mutant plant enzyme polypeptide or mutant stearoyl-acyl carrier protein (ACP) desaturase polypeptide or recombinantly engineered to heterologously produce the polypeptide is provided herein. The host plant may be recombinantly engineered to overproduce the mutant plant enzyme polypeptide or mutant stearoyl-acyl carrier protein (ACP) desaturase polypeptide. In an embodiment, the plant is a castor plant or other seed oil plant. Suitable seed oil plants are known and available to one skilled in the art, including as described herein. In an embodiment, a seed oil plant is selected that is capable of being genetically engineered and recombinantly manipulated to produce or overproduce the mutant polypeptide.

The invention provides a genetically modified eukaryotic host cell which is genetically modified with a nucleic acid encoding a mutant plant enzyme polypeptide or mutant stearoyl-acyl carrier protein (ACP) desaturase polypeptide as provided herein. In an embodiment, the host cell produces vicinal diol. In an embodiment, the host cell produces erythro 9,10 dihydroxy stearate.

The host cell may be any suitable type of cell, including a prokaryotic cell or a eukaryotic cell. In one embodiment the host cell is selected from a bacterial cell, a yeast cell, a fungal cell, an insect cell, algal cell, and a plant cell. In a particular embodiment the host cell is a plant cell. The host cell may be a suitable bacterial cell, yeast cell, fungal cell, an animal cell or a plant cell. In a particular embodiment, the host cell is a bacterial cell.

The invention includes methods for producing a vicinal diol fatty acid in a host cell, the method comprising: a) introducing into a host cell at least one nucleic acid encoding a mutant plant enzyme polypeptide or a mutant stearoyl-acyl carrier protein (ACP) desaturase as provided herein or otherwise engineering the host cell to produce a mutant plant enzyme polypeptide or mutant stearoyl-acyl carrier protein (ACP) desaturase as described and provided herein; and b) culturing the host cell in order to express the mutant plant enzyme polypeptide or mutant stearoyl-acyl carrier protein (ACP) desaturase. The invention includes methods for producing a vicinal diol fatty acid in a host cell, the method comprising: a) introducing into a host cell at least one nucleic acid encoding a mutant stearoyl-acyl carrier protein (ACP) desaturase as provided herein or otherwise engineering the host cell to produce a mutant stearoyl-acyl carrier protein (ACP) desaturase as described and provided herein; and b) culturing the host cell in order to express the mutant stearoyl-acyl carrier protein (ACP) desaturase. In an embodiment, the mutant plant enzyme polypeptide is a polypeptide having an amino acid sequence that is at least 85% identical or at least 90% identical to the sequence of the castor mutant plant enzyme polypeptide, particularly the sequence of SEQ ID NO: 1, 2 or 7.

In a further embodiment, methods are provided for producing erythro 9,10 dihydroxy stearate in a host cell, the method comprising: a) introducing into a host cell at least one nucleic acid encoding a mutant stearoyl-acyl carrier protein (ACP) desaturase as provided herein or otherwise engineering the host cell to produce a mutant stearoyl-acyl carrier protein (ACP) desaturase hereof, and introducing a substrate for the stearoyl-acyl carrier protein (ACP) desaturase enzyme; and b) culturing the host cell in order to express the modified stearoyl-acyl carrier protein (ACP) desaturase, whereby the substrate is converted to erythro 9,10 dihydroxy stearate.

The invention further provides a plant expressing the mutant plant enzyme polypeptide or the mutant stearoyl-acyl carrier protein (ACP) desaturase of the invention. The invention further provides a plant comprising a plant cell of the invention. In one aspect the invention provides a plant comprising a construct of the invention. In an aspect the invention provides a plant genetically modified to comprise or to express a polynucleotide of the invention. In a further embodiment the plant expresses a mutant stearoyl-acyl carrier protein (ACP) desaturase provided herein and encoded by the polynucleotide or nucleic acid of the invention.

The invention further provides a bacterial cell expressing the mutant plant enzyme polypeptide or the mutant stearoyl-acyl carrier protein (ACP) desaturase of the invention. In one aspect the invention provides a bacterial cell comprising a construct of the invention. In an aspect the invention provides a bacterial cell genetically modified to comprise or to express a polynucleotide of the invention. In a further embodiment the bacterial cell expresses a mutant plant enzyme polypeptide or mutant stearoyl-acyl carrier protein (ACP) desaturase provided herein and encoded by the polynucleotide or nucleic acid of the invention.

In a further aspect the invention provides a composition comprising a mutant plant enzyme polypeptide or mutant stearoyl-acyl carrier protein (ACP) desaturase of the invention. In one embodiment the composition comprises the mutant plant enzyme polypeptide or mutant stearoyl-acyl carrier protein (ACP) desaturase and a suitable carrier.

The mutant plant enzyme polypeptide or mutant stearoyl-acyl carrier protein (ACP) desaturase(s) may be modified naturally occurring plant enzyme polypeptide or stearoyl-acyl carrier protein (ACP) desaturase(s). The plants from which the un-modified or naturally occurring mutant plant enzyme polypeptide or stearoyl-acyl carrier protein (ACP) desaturase sequences are derived may be from any plant species that contains the applicable plant enzyme polypeptide at least 85% or at least 90% identical to castor stearoyl-acyl carrier protein (ACP) desaturase or that contains stearoyl-acyl carrier protein (ACP) desaturase and polynucleotide sequences encoding the plant enzyme polypeptide or the stearoyl-acyl carrier protein (ACP) desaturase. The plant cells in which the mutant stearoyl-acyl carrier protein (ACP) desaturase are expressed may be from any plant species. The plants in which the mutant plant enzyme polypeptide or mutant stearoyl-acyl carrier protein (ACP) desaturase are expressed may be from any plant species. In one embodiment the plant cell or plant, is derived from a gymnosperm plant species. In a further embodiment the plant cell or plant, is derived from an angiosperm plant species. In a further embodiment the plant cell or plant, is derived from a from dicotyledonous plant species. In a further embodiment the plant cell or plant, is derived from a monocotyledonous plant species. The plant or plant cell may be seed oil producing plant. The plant or plant cell may be a castor plant cell.

In one embodiment the plant accumulates more vicinal diol in its non-photosynthetic tissues/organs than does a control plant. In a further embodiment the plant accumulates at least 10%, more preferably at least 15%, more preferably at least 20%, more preferably at least 25%, more preferably at least 30%, more preferably at least 40%, more preferably at least 50%, more preferably at least 60%, more preferably at least 80%, more preferably at least 100% more vicinal diol in its non-photosynthetic tissues/organs than does a control plant.

In one embodiment the plant accumulates more 9,10 dihydroxy stearate in its non-photosynthetic tissues/organs than does a control plant. In a further embodiment the plant accumulates at least 10%, more preferably at least 15%, more preferably at least 20%, more preferably at least 25%, more preferably at least 30%, more preferably at least 40%, more preferably at least 50%, more preferably at least 60%, more preferably at least 80%, more preferably at least 100% more 9,10 dihydroxy stearate in its non-photosynthetic tissues/organs than does a control plant.

Suitable control plants include non-transformed or wild-type versions of plant of the same variety and/or species as the transformed plant used in the method of the invention. Suitable control plants also include plants of the same variety and or species as the transformed plant that are transformed with a control construct. Suitable control plants also include plants that have not been transformed with a polynucleotide encoding a mutant stearoyl-acyl carrier protein (ACP) desaturase provided herein. Suitable control plants also include plants that do not express a mutant stearoyl-acyl carrier protein (ACP) desaturase provided herein.

The relative terms, such as increased and reduced as used herein with respect to plants, are relative to a control plant. Suitable control plants include non-transformed or wild-type versions of plant of the same variety and/or species as the transformed plant used in the method of the invention. Suitable control plants also include plants of the same variety and/or species as the transformed plant that are transformed with a control construct. Suitable control constructs include empty vector constructs, known to those skilled in the art. Suitable control plants also include plants that have not been transformed with a polynucleotide encoding a mutant plant enzyme polypeptide or modified stearoyl-acyl carrier protein (ACP) desaturase. Suitable control plants also include plants that do not express a mutant plant enzyme polypeptide or modified stearoyl-acyl carrier protein (ACP) desaturase including at least one amino acid substitution at amino acid 117 and/or 280 or the corresponding amino acid residue.

The term “biomass” refers to the size and/or mass and/or number of vegetative organs of the plant at a particular age or developmental stage. Thus a plant with increased biomass has increased size and/or mass and/or number of vegetative organs than a suitable control plant of the same age or at an equivalent developmental stage. Increased biomass may also involve an increase in rate of growth and/or rate of formation of vegetative organs during some or all periods of the life cycle of a plant relative to a suitable control. Thus increased biomass may result in an advance in the time taken for such a plant to reach a certain developmental stage.

The terms “seed yield”, “fruit yield” and “organ yield” refer to the size and/or mass and/or number of seed, fruit or organs produced by a plant. Thus a plant with increased seed, fruit or organ yield has increased size and/or mass and/or number of seeds, fruit or organs respectively, relative to a control plant at the same age or an equivalent developmental stage.

The polynucleotide molecules of the invention can be isolated by using a variety of techniques known to those of ordinary skill in the art. By way of example, such polypeptides can be isolated through use of the polymerase chain reaction (PCR) described in Mullis et al., Eds. 1994 The Polymerase Chain Reaction, Birkhauser, incorporated herein by reference. The polypeptides of the invention can be amplified using primers, as defined herein, derived from the polynucleotide sequences of the invention.

Further methods for isolating polynucleotides of the invention include use of all, or portions of, the polypeptides having the sequence set forth herein as hybridization probes. The technique of hybridizing labelled polynucleotide probes to polynucleotides immobilized on solid supports such as nitrocellulose filters or nylon membranes, can be used to screen the genomic or cDNA libraries. 1987).

It may be beneficial, when producing a transgenic plant from a particular species, to transform such a plant with a sequence or sequences derived from that species. The benefit may be to alleviate public concerns regarding cross-species transformation in generating transgenic organisms. Additionally when down-regulation of a gene is the desired result, it may be necessary to utilize a sequence identical (or at least highly similar) to that in the plant, for which reduced expression is desired. For these reasons among others, it is desirable to be able to identify and isolate orthologues of a particular gene in several different plant species.

The invention further provides plant cells which comprise a genetic construct of the invention, and plant cells modified to alter expression of a polynucleotide or polypeptide of the invention, or used in the methods of the invention. Plants comprising such cells also form an aspect of the invention.

Methods for transforming plant cells, plants and portions thereof with polypeptides are described in Draper et al., 1988, Plant Genetic Transformation and Gene Expression. A Laboratory Manual. Blackwell Sci. Pub. Oxford, p. 365; Potrykus and Spangenburg, 1995, Gene Transfer to Plants. Springer-Verlag, Berlin; and Gelvin et al., 1993, Plant Molecular Biol. Manual. Kluwer Acad. Pub. Dordrecht. A review of transgenic plants, including transformation techniques, is provided in Galun and Breiman, 1997, Transgenic Plants. Imperial College Press, London.

A number of plant transformation strategies are available (e.g. Birch, 1997, Ann Rev Plant Phys Plant Mol Biol, 48, 297, Hellens R P, et al (2000) Plant Mol Biol 42: 819-32, Hellens R et al Plant Meth 1: 13). For example, strategies may be designed to increase expression of a polynucleotide/polypeptide in a plant cell, organ and/or at a particular developmental stage where/when it is normally expressed or to ectopically express a polynucleotide/polypeptide in a cell, tissue, organ and/or at a particular developmental stage which/when it is not normally expressed. The expressed polynucleotide/polypeptide may be derived from the plant species to be transformed or may be derived from a different plant species. Transformation strategies may be designed to reduce expression of a polynucleotide/polypeptide in a plant cell, tissue, organ or at a particular developmental stage which/when it is normally expressed. Such strategies are known as gene silencing strategies.

Exemplary terminators that are commonly used in plant transformation genetic construct include, e.g., the cauliflower mosaic virus (CaMV) 35S terminator, the Agrobacterium tumefaciens nopaline synthase or octopine synthase terminators, the Zea mays zein gene terminator, the Oryza sativa ADP-glucose pyrophosphorylase terminator and the Solanum tuberosum PI-II terminator.

Selectable markers commonly used in plant transformation include the neomycin phosphotransferase II gene (NPT II) which confers kanamycin resistance, the aadA gene, which confers spectinomycin and streptomycin resistance, the phosphinothricin acetyl transferase (bar gene) for Ignite (AgrEvo) and Basta (Hoechst) resistance, and the hygromycin phosphotransferase gene (hpt) for hygromycin resistance.

Use of genetic constructs comprising reporter genes (coding sequences which express an activity that is foreign to the host, usually an enzymatic activity and/or a visible signal (e.g., luciferase, GUS, GFP) which may be used for promoter expression analysis in plants and plant tissues are also contemplated. The reporter gene literature is reviewed in Herrera-Estrella et al., 1993, Nature 303, 209, and Schrott, 1995, In: Gene Transfer to Plants (Potrykus, T., Spangenberg. Eds) Springer Verlag. Berline, pp. 325-336.

The following are representative publications disclosing genetic transformation protocols that can be used to genetically transform the following plant species: Rice (Alam et al., 1999, Plant Cell Rep. 18, 572); apple (Yao et al., 1995, Plant Cell Reports 14, 407-412); maize (U.S. Pat. Nos. 5,177,010 and 5,981,840); wheat (Ortiz et al., 1996, Plant Cell Rep. 15, 1996, 877); tomato (U.S. Pat. No. 5,159,135); potato (Kumar et al., 1996 Plant J. 9, 821); cassaya (Li et al., 1996 Nat. Biotechnology 14, 736); lettuce (Michelmore et al., 1987, Plant Cell Rep. 6, 439); tobacco (Horsch et al., 1985, Science 227, 1229); cotton (U.S. Pat. Nos. 5,846,797 and 5,004,863); grasses (U.S. Pat. Nos. 5,187,073 and 6,020,539); peppermint (Niu et al., 1998, Plant Cell Rep. 17, 165); citrus plants (Pena et al., 1995, Plant Sci. 104, 183); caraway (Krens et al., 1997, Plant Cell Rep, 17, 39); banana (U.S. Pat. No. 5,792,935); soybean (U.S. Pat. Nos. 5,416,011; 5,569,834; 5,824,877; 5,563,04455 and 5,968,830); pineapple (U.S. Pat. No. 5,952,543); poplar (U.S. Pat. No. 4,795,855); monocots in general (U.S. Pat. Nos. 5,591,616 and 6,037,522); brassica (U.S. Pat. Nos. 5,188,958; 5,463,174 and 5,750,871); cereals (U.S. Pat. No. 6,074,877); pear (Matsuda et al., 2005, Plant Cell Rep. 24(1):45-51); Prunus (Ramesh et al., 2006 Plant Cell Rep. 25(8):821-8; Song and Sink 2005 Plant Cell Rep. 2006; 25(2):117-23; Gonzalez Padilla et al., 2003 Plant Cell Rep. 22(1):38-45); strawberry (Oosumi et al., 2006 Planta. 223(6):1219-30; Folta et al., 2006 Planta April 14; PMID: 16614818), rose (Li et al., 2003), Rubus (Graham et al., 1995 Methods Mol. Biol. 1995; 44:129-33), tomato (Dan et al., 2006, Plant Cell Reports V25:432-441), apple (Yao et al., 1995, Plant Cell Rep. 14, 407-412), Canola (Brassica napus L.). (Cardoza and Stewart, 2006 Methods Mol. Biol. 343:257-66), safflower (Orlikowska et al, 1995, Plant Cell Tissue and Organ Culture 40:85-91), ryegrass (Altpeter et al, 2004 Developments in Plant Breeding 11(7):255-250), rice (Christou et al, 1991 Nature Biotech. 9:957-962), maize (Wang et al 2009 In: Handbook of Maize pp. 609-639) and Actinidia eriantha (Wang et al., 2006, Plant Cell Rep. 25, 5: 425-31). Transformation of other species is also contemplated by the invention. Suitable methods and protocols are available in the scientific literature.

The term “plant” is intended to include a whole plant, any part of a plant, a seed, a fruit, propagules and progeny of a plant.

The term “propagule” means any part of a plant that may be used in reproduction or propagation, either sexual or asexual, including seeds and cuttings.

The plants of the invention may be grown and either selfed or crossed with a different plant strain and the resulting hybrids, with the desired phenotypic characteristics, may be identified. Two or more generations may be grown to ensure that the subject phenotypic characteristics are stably maintained and inherited. Plants resulting from such standard breeding approaches also form an aspect of the present invention.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### Example 1

In previous work, we identified a triple mutant of the castor (Ricinus communis) stearoyl-Acyl Carrier Protein desaturase (T117R/G188L/D280K) that, in addition to introducing a double bond into stearate to produce oleate, performed an additional round of oxidation to convert oleate to a trans allylic alcohol acid (Whittle et al 2008). To determine the contributions of each mutation, in this work we generated individual castor desaturase mutants carrying residue changes corresponding to those in the triple mutant and investigated their catalytic activities. We observed that T117R, and to a lesser extent D280K, accumulated a novel product, namely erythro-9,10-dihydroxystearate, that we identified via its methyl ester through gas chromatography-mass spectrometry and comparison with authentic standards. The use of ¹⁸O₂ labeling showed that the oxygens of both hydroxyl moieties originate from molecular oxygen rather than water. Incubation with an equimolar mixture of ¹⁸O₂ and ¹⁶O₂ demonstrated that both hydroxyl oxygens originate from a single molecule of O₂, proving the product is the result of dioxygenase catalysis. Using prolonged incubation, we discovered that wild-type castor desaturase is also capable of forming erythro-9,10-dihydroxystearate, which presents a likely explanation for its accumulation to ˜0.7% in castor oil, the biosynthetic origin of which had remained enigmatic for decades. In summary, the findings presented here expand the documented constellation of di-iron enzyme catalysis to include a dioxygenase reactivity in which an unactivated alkene is converted to a vicinal diol.

The soluble class of desaturase enzymes exemplified by the castor (Ricinus communis) Δ⁹18:0-ACP desaturase (Lindqvist, 2001) has been shown to contain members that display a variety of chain-length specificities and regioselectivities (Shanklin et al., 2009). Mechanisms have been proposed for both chain length specificity (Cahoon et al., 1997; Whittle and Shanklin, 2001) and for regioselectivity (Guy et al., 2011). During studies on regioselectivity, a triple mutant of the castor acyl-ACP desaturase (T117R/G188L/D280K) was engineered that converts stearoyl-ACP into an allylic alcohol trans-isomer (E)-10-18:1-9-OH via a (Z)-9-18:1 intermediate (Whittle et al., 2008). This was reported as a soluble desaturase acting as an olefin oxygenase similar in behavior to that displayed by another soluble diiron protein, methane monooxygenase (Gherman et al., 2004). It was shown that the conversion of (Z)-9-18:1 substrate to (E)-10-18:1-9-OH product by castor desaturase T117R/G188L/D280K proceeds via hydrogen abstraction at C-11 and highly regioselective hydroxylation (>97%) at C-9 (Whittle et al., 2008). ¹⁸O-labeling studies show that the hydroxyl oxygen in the reaction product is exclusively derived from molecular oxygen.

Experiments were designed to evaluate the individual contributions of each of the castor desaturase amino acid variants T117R, G188L and D280K to allylic alcohol formation. During these experiments, a novel dioxygenase reactivity of the soluble desaturase was discovered that results in the conversion of oleoyl-ACP to erythro-9, 10-dihydroxystearate. Castor desaturase variants T117R and D280K accumulated a product, erythro-9,10-dihydroxystearate that is identified as the methyl ester by gas chromatography/mass spectrometry and analytical comparisons. The use of ¹⁸O₂ labeling shows that the oxygens of both hydroxyl moieties originate from molecular oxygen and not water. Incubation with an equimolar mixture of ¹⁸O₂ and ¹⁶O₂ demonstrates that both hydroxyl oxygens originate from the same molecule of O₂ such that the product is a result of dioxygenase catalysis. The same product was found in TMS-derivatized methyl esters from castor seed where it constitutes approximately 0.7% of the total fatty acids.

### Materials and Methods

### Mutant Construction

Synthesis of the triple mutant T117R/G188L/D280K and D280K single mutants were previously described (Whittle et al., 2008; Guy et al., 2011). The single mutants T117R and G188L were identified by mutagenesis-selection experiments (Whittle and Shanklin, 2001). The open reading frames were introduced into pET9d using XbaI and EcoRI restriction sites and the resulting clones were validated by sequencing.

### Mutant Analysis

Desaturases, and variants thereof, were overexpressed in E. coli BL21(DE3) with the use of pET9d. Recombinant desaturase was enriched to >90% purity by 20CM cation exchange chromatography (Applied Biosystems). Desaturation reactions (600 μl) (Cahoon and Shanklin, 2000) were performed by incubation of the desaturase with 18:0- and 18:1-ACP substrates in the presence of recombinant spinach ACP-I (Beremand et al., 1987). Uniformly deuterated stearate was obtained from Cambridge Isotope Laboratories, Andover Mass., and 9,10 d2 oleate and 11, 11 d2 oleate was obtained from the collection of Tulloch (Tulloch, 1983). Experiments reported herein were replicated three or more times and representative results are presented.

### Fatty Acid Analysis

Fatty acid methyl esters (FAMEs) were prepared by addition of 2 ml of 1% (v/v) NaOCH₃ in methanol and incubated for 60 min at 50° C. Fatty acid methyl esters were extracted twice into 2 ml hexane after acidification with 100 μl of glacial acetic acid. Hexane was evaporated to dryness under a stream of N₂, and samples were resuspended in hexane for GC analysis. FAMEs were dried and resuspended in 100 μl of N,O-bis(trimethylsilyl) trifluoroacetamide (BSTFA)+trimethyl chlorosilane (TMCS) (Supelco) for 45 min at 60° C. to create trimethyl silyl derivatives. Samples were analyzed with an HP5890 gas chromatograph (Agilent) fitted with a 60 m×250 μm SP-2340 capillary column (Supelco). The oven temperature was raised from 100° C. to 160° C. at a rate of 25° C. min-1, and from 160° C. to 240° C. at a rate of 10° C. min-1 with a flow rate of 1.1 ml min-1. Mass spectra were analyzed using an HP5973 mass selective detector (Agilent). For ¹⁸O experiments, oxygen was removed from the sample cell by repeated evacuation and purging of the cell with O₂-free argon using a Schlenk line. Two mixtures were prepared—one containing desaturase enzyme, buffer, ferredoxin NADPH⁺ reductase and substrate, the other containing ferredoxin and NADPH. The two anaerobic mixtures were transferred to sealed reaction vials containing an atmosphere composed of either ¹⁶O₂, ¹⁸O₂ (Cambridge Isotope Laboratories, Andover Mass.), or an equimolar mixture of ¹⁶O₂ and ¹⁸O₂. Reactions were terminated by the addition of toluene, and fatty acids were esterified and silylated as described above for experiments designed to fragment the fatty acid to reveal the position of the vicinal hydroxyl groups. Alternatively, for the labelled oxygen experiments designed to determine the reaction mechanism, fatty acids were converted to methyl esters after which vicinal hydroxy groups were converted to their acetonide derivatives (Singh et al., 2008). To achieve this, methyl ester samples were dried under nitrogen and resuspended in 40 μl of 4 mM ZrCl₄ catalyst in diethyl ether, 200 μl dichloromethane (CH₂Cl₂), and 5 ul dimethoxypropane. The mixture was incubated with shaking at 22° C. for 2 hrs. The mixture was extracted with 3 ml chloroform (CHCl₃) and 1 ml water, separated by centrifugation (at 1,500 g for 5 min) and the lower phase was collected and dried under nitrogen before resuspension in hexane for GC/MS analysis. Samples were analyzed on HP6890/5973 GC/MS equipped with a 30 m×250 μm HP 5MS capillary column (Supelco). Oven temperature was held at 100° C. for 2 min, raised to 300° C. at the rate of 20° C. min-1 and held for 2 min.

### Accession Numbers

Sequence data can be found in the GenBank/EMBL data libraries under accession number M59857.

### Results

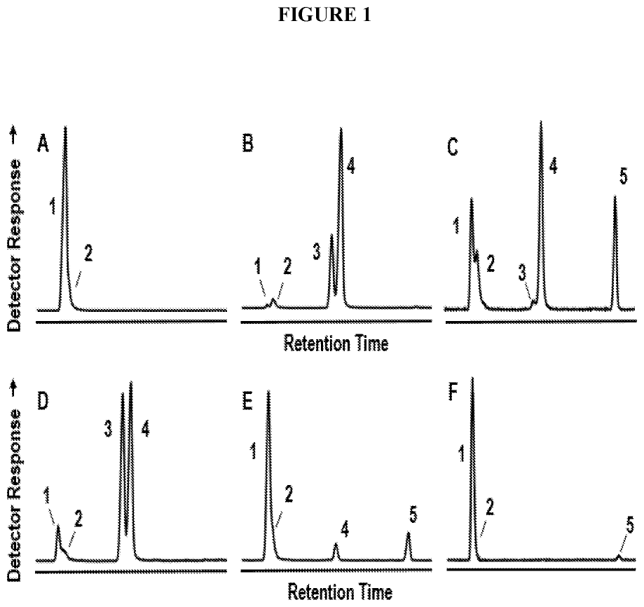

As part of a continuing structure-function analysis of diiron enzymes, the contributions were analyzed of each of the mutations within the castor T117R/G188L/D280K triple mutant that converts oleoyl-ACP into (E)-10-18: 1-9-OH (Whittle et al., 2008). Each of the individual mutants was constructed and tested for its activity using oleoyl-ACP as a substrate. In each case, the product profiles were determined by GC-MS analysis. The results are shown in FIG. 1. The GC elution profile of the substrate is shown in Panel A (FIG. 1A) and features a peak corresponding to 18:1Δ9 methyl ester (peak 1). A minor shoulder peak can be attributed to 18:1Δ11 (peak 2) and is a well-known artifact of the expression system. As shown in Panel B (FIG. 1B), the triple mutant T117R/G188L/D280K converted most of the oleoyl-ACP substrate into a mixture of the Z(cis)18:1Δ10 9OH (peak 3) and E(trans) 18:1Δ10 9OH allylic alcohol (peak 4) isomers, with the E form predominating by approximately 3-fold over the Z form.

### Reactivity of the Castor Desaturase Single Mutants T117R, G188L, and D280K

Each of the single mutants was found to be active with respect to the oleoyl-ACP substrate (FIGS. 1C, 1D and 1E). The T117R mutant produced approximately 15-fold more of the E 18:1Δ10 9OH isomer than the corresponding Z isomer. However, a new peak (labeled 5 in FIG. 1C) became apparent at an elution time that was not characteristic of the silylated derivatives of commonly occurring fatty acid methyl esters. The G188L mutant produced approximately a 1:1 mixture of E and Z isomers of 18:1Δ10 9OH (FIG. 1D), but no detectable traces of the novel fatty acid species (peak 5) produced by the T117R mutant. The D280K mutant was less active than T117R and G188L, producing only a small amount of the E isomer of 18:1Δ10 9OH (FIG. 1E), along with a small amount of the novel fatty acid (peak 5). The wild type desaturase showed very little activity with its natural product oleoyl-ACP, but close inspection revealed the production of a trace of novel species (5) based on its elution time and mass spectra (FIG. 1F).

### The Novel Fatty Acid Product (5) is 9,10-Dihydroxystearate

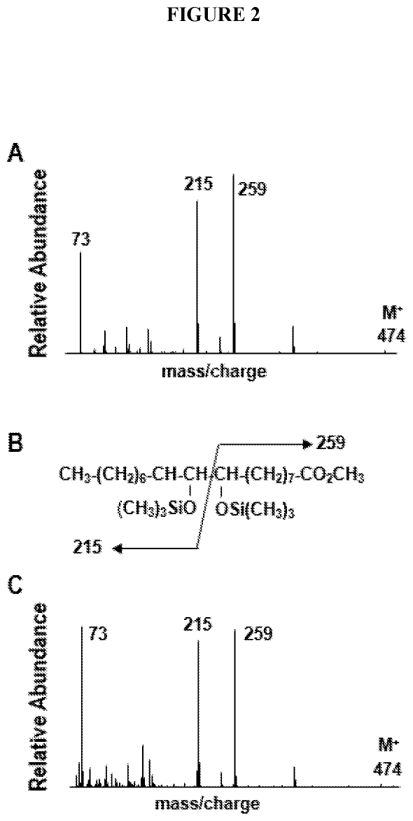

Mass spectral analysis of the product peak 5 produced by the T117R mutant (FIG. 1C) revealed a molecular ion of 474 AMU, consistent with an 18C fatty acid methyl ester containing two silylated hydroxyl groups (FIG. 2A). Fragmentation of the product peak between the two silyl groups produced fragments of 259 AMU for the carboxyl-containing fragment and 215 AMU for the methyl-containing fragment (diagrammed in FIG. 2B), consistent with the presence of vicinal hydroxyl groups at C9 and C10. The identify of peak 5 was confirmed by comparison of its fragmentation pattern with that of a silylated authentic commercial standard of erythro-methyl 9,10-dihydroxy stearate (FIG. 2C). Analysis of peak 5 from the D280K mutant also showed the same fragmentation pattern.

### 9,10-Dihydroxystearate Produced by the T117R Mutant is Solely in the Erythro Configuration

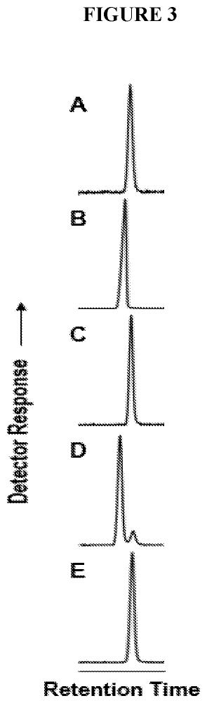

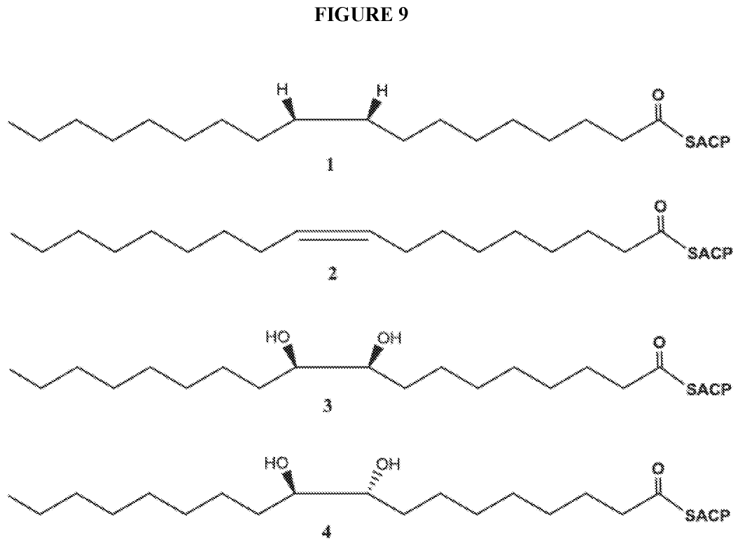

Fatty acids containing vicinal mid-chain hydroxyl groups may exist as threo or erythro diastereoisomers (FIG. 9). To distinguish between these possibilities, we compared the GC elution times of the present fatty acid product from T117R with those of authentic threo and erythro-9, 10-dihydroxystearate standards (FIGS. 3, A, B, and C, respectively). The T117R product eluted as a single defined peak without any detectable shoulders (FIG. 3A) and coeluted with authentic erythro standard (FIG. 3C). The authentic threo standard (FIG. 3B) eluted ahead of that of the T117R product (FIG. 3A). When a small amount of the T117R product was mixed with either the threo standard (FIG. 3D), or the erythro standard (FIG. 3E), two peaks can be seen for the sample spiked with threo standard, whereas a single coeluting peak can be seen for the spiked erythro standard. These results confirm the assignment of the T117R product as erythro-9, 10-dihydroxystearate.

The Hydroxyl Oxygens at Both C9 and C10 are Derived from Molecular Oxygen

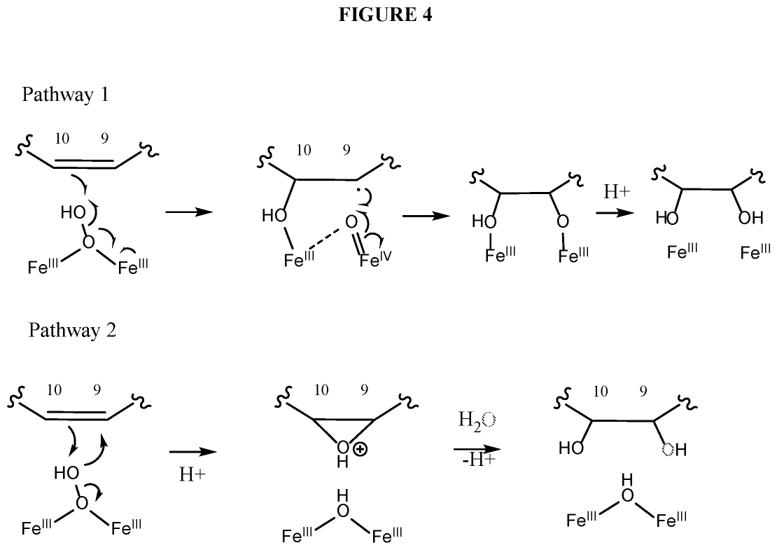

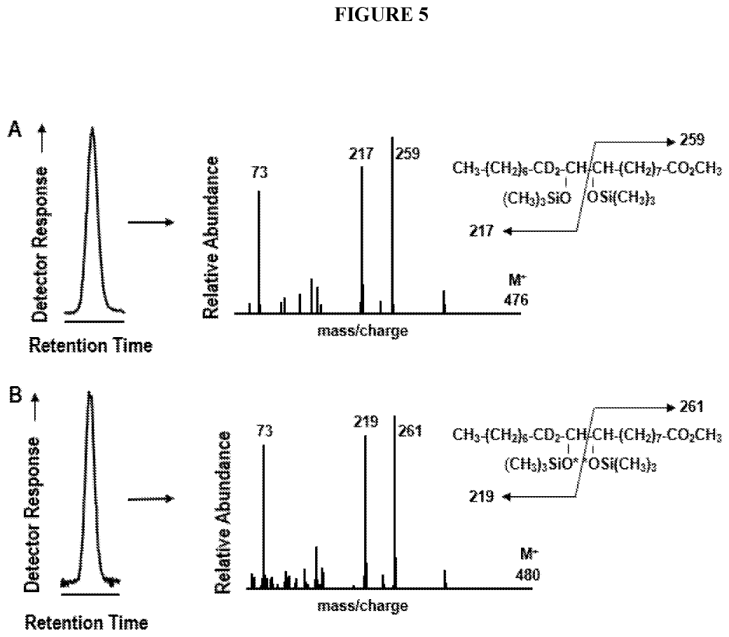

The oxygen atoms in either of the two hydroxyl groups could in principle arise from water or molecular oxygen (FIG. 4). To distinguish between these possibilities, T117R, oleoyl-ACP, and all assay components were first degassed by multiple gas exchange cycles employing vacuum and 02-free argon with the use of a Schlenk line (Arnold and Bohle, 1996) to remove residual atmospheric ¹⁶O₂ from the sealed reaction vials. Assay reactions were subsequently incubated in the presence of ¹⁶O₂ or ¹⁸O₂. Mass-labeled 18:1 d₂-11,11 oleoyl-ACP were used for these assays to ensure the product observed was derived from the enzymatic reaction rather than from endogenous oleate contaminant. Analysis of the methylated silylated products from reaction under air yielded the expected 217 and 259 AMU products (the methyl fragment increased by 2 AMU relative to unlabeled product results from the substitution for the two hydrogens at C11 for deuterons (FIG. 5A). The same experiment performed under ¹⁸O₂ resulted in the production of fragments of 219 and 261 AMU, consistent with the incorporation of one ¹⁸O at each of the hydroxyl positions.

The Formation of 9, 10-Dihyroxystearate from Oleate is the Result of a Dioxygenase Reaction

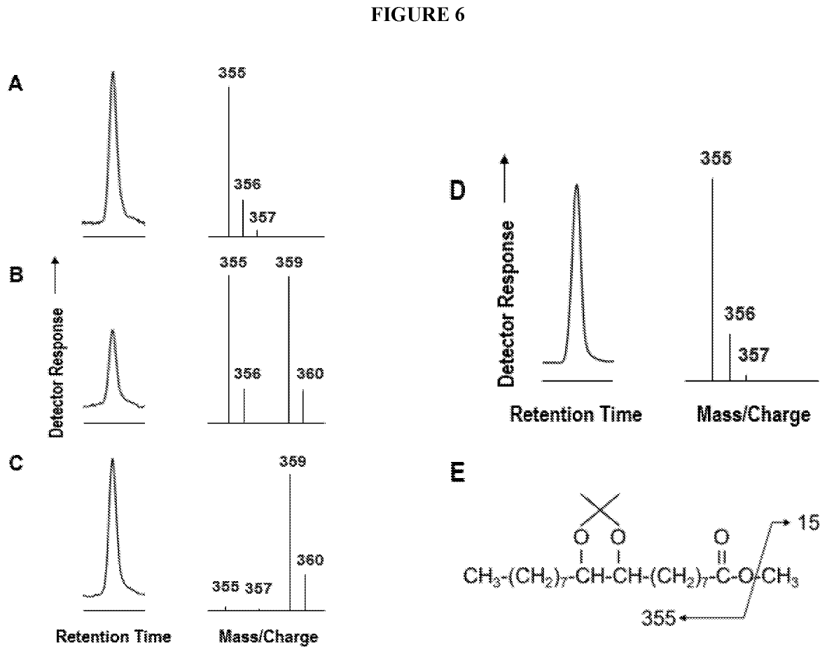

The incorporation of molecular oxygen at the 9 and 10 positions of oleate could in principle result from a single dioxygenase reaction, or from two sequential monooxygenase reactions. To distinguish between these possibilities, samples were degassed as described above. A reaction was performed under an atmosphere containing an equimolar fraction of ¹⁶O₂ and ¹⁸O₂ (FIG. 6 B) and mass spectrometry was performed on methylated acetonide derivatives of the product (FIG. 6E). Acetonide derivatives were used because they protect vicinal hydroxy groups while maximizing the detectable mass ion of the product. If the reaction operates via a dioxygenase mechanism, then the oxygen atoms at both hydroxyl positions may derive exclusively from either ¹⁶O₂ or ¹⁸O₂, resulting in either M or M+4 species. Alternatively, if the mechanism employs two sequential monooxygenase reactions, a 1:2:1 pattern of M:M+2:M+4 would be expected by random incorporation of either ¹⁶O or ¹⁸O at each hydroxyl position. Consistent with a dioxygenase mechanism, reactions performed under an equimolar mix of ¹⁶O₂ and ¹⁸O₂ yielded only M and M+4 peaks (355 and 359), with no detectable 357 species (FIG. 6B). Individually controlled ¹⁶O₂ and ¹⁸O₂ reactions showed the expected 355 and 359 major species accompanied by minor peaks at M+1 and M+2 that approximate the natural abundance of ¹³C (FIGS. 6, A and C, respectively). That M+1 and M+2 peaks originate from natural ¹³C was confirmed by the fragmentation of equivalent derivatives of an authentic erythro-9, 10-dihydroxystearate, which showed the same proportions of M, M+1 and M+2 species (FIG. 6D).

### The Native Castor Desaturase can Convert Oleoyl-ACP to 9,10-Dihydroxystearate

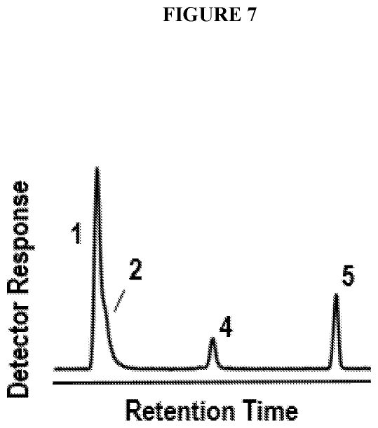

The formation of dihydroxystearate with selected mutated desaturases prompted a probe for the formation of this compound by wild-type enzyme. Interestingly, using a prolonged time of incubation (240 min) with oleoyl-ACP as substrate, it was possible to identify production of 9, 10-dihydroxystearate (5) at low levels (FIG. 7). This compound was accompanied by lesser amounts of E 18:1Δ10 9 OH (4).

### Castor Oil Contains Erythro-9,10-Dihydroxystearate

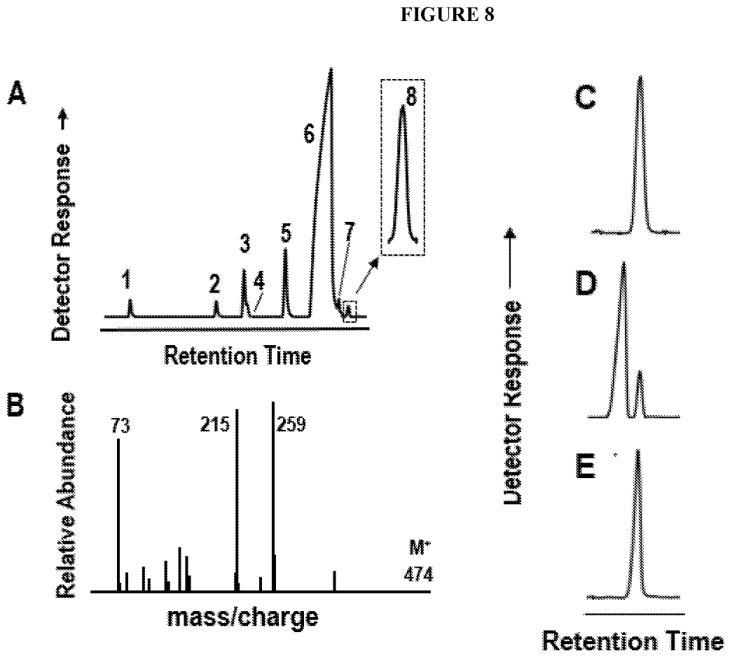

The observation that the native castor desaturase can produce small amounts of 9,10-dihydroxystearate (5) correlates well with an early report by King et al (King, 1942) where a small amount of 9,10-dihydroxystearate (5) from castor oil was isolated. To confirm this observation a fatty acid extract of castor seeds was analyzed by GC-MS after methylation and silylation. Chromatograms of castor seed fatty acid derivatives (FIG. 8A) showed the expected common C16 and C18 fatty acids, along with a major peak of ricinoleic acid which is followed by a small discrete peak (labeled 8 in FIG. 8A inset) of approximately 0.7% (of total fatty acids), which corresponds to the elution time of disilylated methyl 9, 10-dihydroxystearate. Mass spectral analysis of this peak revealed fragments of 215 and 259 AMU confirming its assignment as 9, 10-dihydroxystearate (compare FIG. 8B with FIGS. 2A and C). Based on the in vitro assays using purified enzyme reported above, it can be hypothesized that 9,10 dihydroxystearate arises from the dioxygenation of oleoyl-ACP product of the stearoyl-ACP desaturase. In such a case, the 9,10-dihydroxystearate would be in the erythro form as originally proposed (Morris and Crouchman, 1972). Therefore coelution studies were conducted with authentic threo or erythro standards (FIG. 8, C-E). The 9, 10 dihydroxystearate isolated from castor bean eluted as a single peak (FIG. 8C) with the same or similar mobility as that of the authentic erythro standard (FIG. 8E). In contrast, two peaks are seen in the spiking experiment using threo standard (FIG. 8D).

### Discussion

Stereoselective dihydroxylation reactions are important to the chemical industry (Borrell and Costas, 2017) because diols serve as valuable synthons. The osmium based asymmetric dihydroxylation reaction (Crispino and Sharpless, 1993) is a prominent example of controlled olefin oxidation and was (in part) recognized by the award of the 2001 Nobel Prize in Chemistry to its inventor, Karl B. Sharpless. In addition, biocatalytic diol formation from aromatics by whole-cell mutant Pseudomonas cultures has furnished the synthetic chemist with a variety of enantiomerically pure cyclohexadienecis-diols. (Hudlicky and Thorpe, 1996). Much effort has also been expended to develop iron-based biomimetic catalytic methodology for this reaction (Oloo and Que, 2015). Herein, we report the details of our investigation into a “green chemical approach”: the castor D918:0-ACP desaturase-mediated syn-dihydroxylation of an unactivated alkene in the form of oleoyl-ACP to erythro-9,10-dihydroxystearoyl-ACP.

Stearoyl-ACP desaturase belongs to the nonheme diiron subclass of oxidative enzymes that have been shown to mediate a variety of chemical transformations including dehydrogenation and mono-oxygenation. Typical products include primary, secondary, and allylic alcohols in addition to the conversion of double bonds to epoxides (Wallar and Lipscomb, 1996). However, a diiron center performing dioxygen chemistry to convert a double bond to a vicinal diol as reported here is without precedent. The closest comparable example we are aware of is arylamine oxygenase (Cm1I) from the chloramphenicol biosynthesis pathway, which incorporates two oxygens from O2 into the aryl-nitro product; however, this occurs in two consecutive mono-oxygenations (Komor et al., 2017). We envision the conversion of alkene to vicinal erythro-diol in this work to be mechanistically related (FIG. 4) to that described for Rieske cisdiol-forming dioxygenases (Ensley et al., 1982; Karlsson et al., 2003). More specifically, we envision involvement of a bridged hydroperoxo-di-iron species similar to that proposed by Solomon and Srnec (Chalupský et al., 2014) for the conversion of stearate to oleate by two consecutive hydrogen atom abstractions: “—CH2-CH2-” to “—CH5CH—.” When presented with an alkene moiety, the vinyl hydrogens are unavailable for abstraction for steric reasons and this same species is forced to transfer two oxygen atoms to substrate as shown in FIG. 4 (Pathway 1). Our oxygen-labelling experiments rule out an epoxidation/hydrolysis route (Pathway 2).

It is possible that our T117R mutant may change the molecular architecture of the substrate binding cavity, altering the relative orientation of the substrate with respect to the hydroperoxo-di-iron group and facilitating deoxygenation relative to the wild-type enzyme. That the diol is produced as the erythro diastereoisomer, in which both hydroxy groups occur on one face (FIG. 3), is consistent with the geometry of the active site substrate binding cavity with respect to the di-iron active site oxidant (Lindqvist et al., 1996), in which stearate binds in a quasi-eclipsed conformation at C9 and C10, projecting the pro-(R) hydrogens toward the active site oxidant (Behrouzian et al., 2002). Future availability of a crystal structure of the T117R mutant in complex with bound oleoyl-ACP, or of the T117R mutant alone or with substrate bound as previously modeled (Whittle et al., 2008), would be useful starting points for probing mechanistic models using computational methods such as density functional theory. Indeed, homology modeling was recently shown to be a useful approach for elucidating selectivity mechanisms of desaturase enzymes such as FAD2 and FAD3 (Cai et al., 2018).

The low or insufficient levels of 9, 10 dihydroxystearate naturally in castor suggest that the natural system is not optimized to produce this particular product. Higher levels of the diol may accumulate via enzymes with active site geometries that permit more efficient dioxygenation. Cardimine impatiens is an example of a plant that may accumulate approximately 25% of 9, 10-dihydroxystearate (and its chain-elongation products) in its seed oil (Mikolajczak et al., 1964). It may contain a desaturase that has undergone mutation/selection to optimize the production of the diol from the initial alkene product. Examples of desaturases with multiple sequential oxidation activity include Hedera helix (English ivy) which can perform Δ9-followed by Δ4 desaturation on stearoyl-ACP (Guy et al., 2007); FM1, a fungal membrane desaturase that sequentially inserts a Δ12 followed by a Δ15 double bond into oleoyl-phosphatidyl ethanolamine (oleoyl-PE) (Cai et al., 2018); and an insect multifunctional enzyme that functions as a Δ11 desaturase, Δ11 acetylenase and Δ13 desaturase (Serra et al., 2007).

Oxygenated fatty acids such as ricinoleic- and vernolic acids are typically produced in the endoplasmic reticulum by variant FAD2 membrane-bound desaturases (van de Loo et al., 1995; Lee et al., 1998). On the other hand, fatty acids with unusual double bond positions such as 16:1Δ4, 16:1Δ9, 18:1 Δ6 may be synthesized within the plastid (Shanklin and Cahoon, 1998). Thus, the present production of oxygenated fatty acids such as the erythro-9, 10-dihydroxystearate in the plastid as described herein is very unusual and unique. There could be a variant acyl-ACP thioesterase that cleaves the vicinal diol fatty acid from its ACP adduct in addition to specialized acyltransferases and other components that facilitate its transfer from the plastid to triglyceride storage lipids in species with high/higher levels of accumulation such as C. impatiens.

9,10-dihydroxystearate was reported as a component of castor oil (King, 1942) at approximately 1% of the total fatty acids (Sreenivasan et al., 1956) many decades ago. The stereochemistry of the diol was determined to be the erythro configuration (Morris and Crouchman, 1972). Castor oil samples evaluated in the vicinal diol fatty acid work described here contained approximately 0.7% of erythro-9,10-dihydroxystearate. Therefore, the wild type castor desaturase can produce this compound, but in low amounts which are not suitable for applications. In addition, the present results indicate the plasticity of the non-heme diiron catalytic center in the desaturase family of enzymes. Subtle changes in the active site architecture of these versatile oxidants provides alters the products produced and also may allow new reaction pathways. Mechanistic work may further indicate a relationship between reaction outcome and active site architecture.

### REFERENCES

Arnold E V, Bohle D S (1996) Isolation and oxygenation reactions of nitrosyl myoglobins. Methods Enzymol 269:41-55
Bai Y, McCoy J G, Levin E J, Sobrado P, Rajashankar K R, Fox B G, Zhou M (2015) X-ray structure of a mammalian stearoyl-CoA desaturase. Nature 524: 252-256
Behrouzian B, Savile C K, Dawson B, Buist P H, Shanklin J (2002) Exploring the hydroxylation-dehydrogenation connection: Novel catalytic activity of castor stearoyl-ACP D(9) desaturase. J Am Chem Soc 124:3277-3283
Beremand P D, Hannapel D J, Guerra D J, Kuhn D N, Ohlrogge J B (1987) Synthesis, cloning, and expression in Escherichia coli of a spinach acyl carrier protein-I gene. Arch. Biochem. Biophys. 256: 90-100
Bhar P, Reed D W, Covello P S, Buist P H (2012) Topological Study of Mechanistic Diversity in Conjugated Fatty Acid Biosynthesis. Angewandte Chemie-International Edition 51: 6686-6690
Bloch K (1969) Enzymatic synthesis of monounsaturated fatty acids. Acc. Chem. Res. 2: 193-202
Borrell M, Costas M (2017) Mechanistically Driven Development of an Iron Catalyst for Selective Syn-Dihydroxylation of Alkenes with Aqueous Hydrogen Peroxide. Journal of the American Chemical Society 139: 12821-12829
Broadwater J A, Whittle E, Shanklin J (2002) Desaturation and hydroxylation. Residues 148 and 324 of Arabidopsis FAD2, in addition to substrate chain length, exert a major influence in partitioning of catalytic specificity. J Biol Chem 277: 15613-15620
Broun P, Shanklin J, Whittle E, Somerville C (1998) Catalytic plasticity of fatty acid modification enzymes underlying chemical diversity of plant lipids. Science 282: 1315-1317
Buist P H (2004) Fatty acid desaturases: selecting the dehydrogenation channel. Nat Prod Rep 21: 249-262.
Cahoon E B, Lindqvist Y, Schneider G, Shanklin J (1997) Redesign of soluble fatty acid desaturases from plants for altered substrate specificity and double bond position. Proc. Natl. Acad. Sci. USA 94: 4872-4877
Cahoon E B, Shanklin J (2000) Substrate-dependent mutant complementation to select fatty acid desaturase variants for metabolic engineering of plant seed oils. Proc Natl Acad Sci USA 97: 12350-12355
Cai Y H, Yu X H, Liu Q, Liu C J, Shanklin J (2018) Two clusters of residues contribute to the activity and substrate specificity of Fm1, a bifunctional oleate and linoleate desaturase of fungal origin. Journal of Biological Chemistry 293: 19844-19853
Chalupsky J, Rokob T A, Kurashige Y, Yanai T, Solomon E I, Rulisek L, Srnec M (2014) Reactivity of the Binuclear Non-Heme Iron Active Site of Delta(9) Desaturase Studied by Large-Scale Multireference Ab Initio Calculations. Journal of the American Chemical Society 136: 15977-15991
Crispino G A, Sharpless K B (1993) Enantioselective Synthesis of Juvenile Hormone-Iii in 3 Steps from Methyl Farnesoate. Synthesis-Stuttgart: 777-779
Edmondson D E, Juynh B H (1996) Diiron cluster intermediates in biological oxygen activation reactions. Inorg. Chimica Acta 252: 399-404
Ensley B D, Gibson D T, Laborde A L (1982) Oxidation of Naphthalene by a Multicomponent Enzyme-System from Pseudomonas Sp Strain Ncib9816. Journal of Bacteriology 149: 948-954
Fox B G, Lyle K S, Rogge C E (2004) Reactions of the diiron enzyme stearoyl-acyl carrier protein desaturase. Acc Chem Res 37: 421-429
Gherman B F, Baik M H, Lippard S J, Friesner R A (2004) Dioxygen activation in methane monooxygenase: A theoretical study. Journal of the American Chemical Society 126: 2978-2990
Guy J E, Whittle E, Kumaran D, Lindqvist Y, Shanklin J (2007) The crystal structure of the ivy Delta4-16:0-ACP desaturase reveals structural details of the oxidized active site and potential determinants of regioselectivity. J Biol Chem 282: 19863-19871
Guy J E, Whittle E, Moche M, Lengqvist J, Lindqvist Y, Shanklin J (2011) Remote control of regioselectivity in acyl-acyl carrier protein-desaturases. Proc Natl Acad Sci USA 108: 16594-16599
Hudlicky T, Thorpe A J (1996) Current status and future perspectives of cyclohexadiene-cis-diols in organic synthesis: Versatile intermediates in the concise design of natural products. Chemical Communications: 1993-2000
Karlsson A, Parales J V, Parales R E, Gibson D T, Eklund H, Ramaswamy S (2003) Crystal structure of naphthalene dioxygenase: Side-on binding of dioxygen to iron. Science 299: 1039-1042
King G (1942) The dihydroxystearitc acid of castor oil—Its constitution and structural relationship to the 9 10-dihydroxystearic acids m p's 132 degrees and 95 degrees. Journal of the Chemical Society: 387-391
Komor A J, Rivard B S, Fan R X, Guo Y S, Que L, Lipscomb J D (2017) CmII N-Oxygenase Catalyzes the Final Three Steps in Chloramphenicol Biosynthesis without Dissociation of Intermediates. Biochemistry 56: 4940-4950
Lee M, Lenman M, Banas A, Bafor M, Singh S, Schweizer M, Nilsson R, Liljenberg C, Dahlqvist A, Gummeson P O, Sjodahl S, Green A, Stymne S (1998) Identification of non-heme diiron proteins that catalyze triple bond and epoxy group formation. Science 280: 915-918
Lindqvist Y (2001) Delta nine stearoyl-acyl carrier protein desaturase. John Wiley & Sons, Chichester UK
Lindqvist Y, Huang W, Schneider G, Shanklin J (1996) Crystal structure of a delta nine stearoyl-acyl carrier protein desaturase from castor seed and its relationship to other diiron proteins. EMBO J. 15: 4081-4092
Mikolajczak K L, Wolff I A, Smith C R (1964) Dihydroxy Fatty Acids in Petroleum Ether Extract of Cardamine Impatiens Seed. Journal of the American Oil Chemists Society 41: 22-&
Morris L J, Crouchman M L (1972) Absolute Optical Configurations of Isomeric 9,10-Epoxystearic, 9,10-Dihydroxystearic and 9,10,12-Trihydroxystearic Acids. Lipids 7: 372-+
Oloo W N, Que L (2015) Bioinspired Nonheme Iron Catalysts for C—H and C═C Bond Oxidation: Insights into the Nature of the Metal-Based Oxidants. Accounts of Chemical Research 48: 2612-2621
Serra M, Pina B, Abad J L, Camps F, Fabrias G (2007) A multifunctional desaturase involved in the biosynthesis of the processionary moth sex pheromone. Proc Natl Acad Sci USA 104: 16444-16449
Shanklin J, Cahoon E B (1998) Desaturation and related modifications of fatty acids. Annu. Rev. Plant Physiol. Plant. Mol. biol. 49: 611-641
Shanklin J, Guy J E, Mishra G, Lindqvist Y (2009) Desaturases—emerging models for understanding functional diversification of diiron-containing enzymes. J Biol Chem 284 18559-18563
Shanklin J, Somerville C (1991) Stearoyl-acyl-carrier-protein desaturase from higher plants is structurally unrelated to the animal and fungal homologs. Proc. Natl. Acad. Sci. USA 88: 2510-2514
Singh S, Duffy C D, Shah S T A, Guiry P J (2008) ZrCl(4) as an efficient catalyst for a novel one-pot protection/deprotection synthetic methodology. Journal of Organic Chemistry 73: 6429-6432
Sreenivasan B, Kamath N R, Kane J G (1956) Studies on Castor Oil. 1. Fatty Acid Composition of Castor Oil. Journal of the American Oil Chemists Society 33: 61-66
Tulloch A P (1983) Synthesis, analysis and application of specifically deuterated lipids. Prog Lipid Res 22: 235-256
van de Loo F J, Broun P, Turner S, Somerville C (1995) An oleate 12-hydroxylase from Ricinus communis L. is a fatty acyl desaturase homolog. Proc. Natl. Acad. Sci. USA 92: 6743-6747
Wallar B J, Lipscomb J D (1996) Dioxygen activation by enzymes containing binuclear non-heme iron clusters. Chem. Rev. 96: 2625-2657
Wang H, Klein M G, Zou H, Lane W, Snell G, Levin I, Li K, Sang B C (2015) Crystal structure of human stearoyl-coenzyme A desaturase in complex with substrate. Nat Struct Mol Biol 22: 581-585
Whittle E, Shanklin J (2001) Engineering delta 9-16:0-acyl carrier protein (ACP) desaturase specificity based on combinatorial saturation mutagenesis and logical redesign of the castor delta 9-18:0-ACP desaturase. J Biol Chem 276: 21500-21505
Whittle E J, Tremblay A E, Buist P H, Shanklin J (2008) Revealing the catalytic potential of an acyl-ACP desaturase: tandem selective oxidation of saturated fatty acids. Proc Natl Acad Sci USA 105: 14738-14743

This invention may be embodied in other forms or carried out in other ways without departing from the spirit or essential characteristics thereof. The present disclosure is therefore to be considered as in all aspects illustrate and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

### INCORPORATION OF SEQUENCE LISTING

Incorporated herein by reference in its entirety is the Sequence Listing for the application. The Sequence Listing is disclosed on a computer-readable ASCII text file titled, “BSA19-17_IP2019-012-02_sequence_listing.txt”, created on Aug. 28, 2020. The sequence_listing.txt file is 45.0 kb in size.

What is claimed is:

1. A mutant plant diiron enzyme polypeptide capable of a dioxygenase reaction mechanism wherein a double bond is converted to a vicinal diol.

2. The mutant of claim 1 which is a mutant stearoyl-acyl carrier protein (ACP) desaturase polypeptide capable of catalyzing the conversion of oleoyl-ACP to erythro 9,10 dihydroxy stearate comprising:
(a) an amino acid replacement of the threonine (T) at amino acid residue 117 of the processed plastid polypeptide sequence and corresponding to residue 117 of SEQ ID NO: 2 or of the amino acid at the corresponding position in a plant stearoyl-ACP desaturase polypeptide;
(b) an amino acid replacement of the aspartic acid (D) at amino acid residue 280 of the processed plastid polypeptide sequence and corresponding to residue 280 of SEQ ID NO: 2 or of the amino acid at the corresponding position in a plant stearoyl-ACP desaturase polypeptide; or
(c) an amino acid replacement of the threonine (T) at amino acid residue 117 of the processed plastid polypeptide sequence and corresponding to residue 117 of SEQ ID NO: 2 or of the amino acid at the corresponding position in a plant stearoyl-ACP desaturase polypeptide and an amino acid replacement of the aspartic acid (D) at amino acid residue 280 of the processed plastid polypeptide sequence and corresponding to residue 280 of SEQ ID NO: 2 or of the amino acid at the corresponding position in a plant stearoyl-ACP desaturase polypeptide.

3. The polypeptide of claim 2, wherein the polypeptide comprises an amino acid replacement at residue 117 or its corresponding position wherein the amino acid threonine or such other hydroxylic amino acid or amino acid having an uncharged polar R group is replaced with a basic amino acid or charged or nonpolar R group.

4. The polypeptide of claim 2, wherein the polypeptide comprises an amino acid replacement at residue 280 or its corresponding position wherein the amino acid aspartic acid or such other acidic amino acid or amino acid having a polar R group is replaced with a basic amino acid or uncharged or nonpolar R group.

5. The polypeptide of claim 2 wherein the polypeptide comprises an amino acid replacement at residue 117 or its corresponding position wherein the amino acid threonine or such other hydroxylic amino acid or amino acid having an uncharged polar R group is replaced with a basic amino acid or charged or nonpolar R group and further comprises an amino acid replacement at residue 280 or its corresponding position wherein the amino acid aspartic acid or such other acidic amino acid or amino acid having a polar R group is replaced with a basic amino acid or uncharged or nonpolar R group.

6. The polypeptide of claim 2 wherein the polypeptide comprises an amino acid replacement at residue 117 or its corresponding position wherein the amino acid threonine is replaced with a basic amino selected from arginine, lysine and histidine.

7. The polypeptide of claim 2 wherein the polypeptide comprises an amino acid replacement at residue 280 or its corresponding position wherein the amino acid aspartic acid is replaced with a basic amino selected from arginine, lysine and histidine.

8. The polypeptide of claim 2 wherein the polypeptide catalyzing the conversion of oleoyl-ACP to erythro 9,10 dihydroxy stearate generates at least 10 fold more erythro 9,10 dihydroxy stearate than the wild type or native, non mutant plant stearoyl-acyl carrier protein (ACP) desaturase polypeptide.

9. An isolated nucleic acid encoding the polypeptide of any of claims 2-8.

10. A host plant recombinantly engineered to produce or overproduce the polypeptide of any of claims 1-8.

11. The host plant of claim 10 wherein the plant is a castor plant or other seed oil plant.

12. A genetically modified eukaryotic host cell which is genetically modified with a nucleic acid encoding a mutant stearoyl-acyl carrier protein (ACP) desaturase polypeptide of any of claims 2-8.

13. The host cell of claim 12, wherein the host cell is a yeast cell, fungal cell, an animal cell or a plant cell.

14. A method for producing a vicinal diol fatty acid in a host cell, the method comprising: a) introducing into a host cell at least one nucleic acid of claim 9 or otherwise engineering the host cell to produce a mutant stearoyl-acyl carrier protein (ACP) desaturase of any of claims 2-8; and b) culturing the host cell in order to express the mutant stearoyl-acyl carrier protein (ACP) desaturase.

15. A method for producing erythro 9,10 dihydroxy stearate in a host cell, the method comprising: a) introducing into a host cell at least one nucleic acid of claim 9 or otherwise engineering the host cell to produce a mutant stearoyl-acyl carrier protein (ACP) desaturase of any of claims 2-8, and introducing a substrate for the stearoyl-acyl carrier protein (ACP) desaturase enzyme; and b) culturing the host cell in order to express the modified stearoyl-acyl carrier protein (ACP) desaturase, whereby the substrate is converted to erythro 9,10 dihydroxy stearate.
